(19) United States
(12) Patent Application Publication
(10) Pub. No.: US 2015/0087553 A1
(43) Pub. Date: Mar. 26, 2015

(54) EARLY TRIMESTER SCREENING FOR EARLY- AND LATE-ONSET PREECLAMPSIA

(71) Applicant: Bioscreening and Diagnostics, LLC, Detroit, MI (US)

(72) Inventors: Ray O. Bahado-Singh, Grosse Pointe Shores, MI (US); Kypros Nicolaides, London (GB)

(21) Appl. No.: 14/394,167

(22) PCT Filed: Apr. 12, 2013
(86) PCT No.: PCT/US2013/036444

§ 371 Date: Oct. 13, 2014

Related U.S. Application Data
(60) Provisional application No. 61/623,757, filed on Apr. 13, 2012.

(51) Int. Cl. G01N 33/68; G01N 33/50

(52) U.S. Cl. CPC G01N 33/689; G01N 33/50; G01N 2800/368; G01N 2800/50
USPC 506/12; 436/65

(57) ABSTRACT

The present invention is directed to methods for predicting a pregnant woman's risk of developing early-onset preeclampsia or late-onset preeclampsia. The methods are based on measuring one or more metabolites obtained from a pregnant woman's bodily fluid, such as blood or urine, which were found to be predictive of early-onset preeclampsia and late-onset preeclampsia.

### FIELD OF THE INVENTION

The present invention generally relates to methods for predicting a pregnant woman's risk of developing early-onset preeclampsia or late-onset preeclampsia based on measuring concentrations of one or more metabolites obtained from a pregnant woman's bodily fluid, such as blood or urine.

### BACKGROUND OF THE INVENTION

Preeclampsia is a disorder of pregnant women characterized by high blood pressure (systolic blood pressure ≧140 mmHg or diastolic blood pressure ≧90 mmHg) occurring after 20 weeks of pregnancy in women with previously normal blood pressure. In addition there is an increased level of proteins in the urine compared to normal. Increased proteinuria is defined as ≧300 mg in a 24 hour collection of urine (The National High Blood Pressure Education Program Working Group Report on High Blood Pressure in Pregnancy. Am J Obstet General 2000; 183:S1-S22). Along with elevated blood pressure, there may be associated signs and symptoms such as headache, abdominal pain, bleeding problems, seizure and complications, such as poor fetal growth, preterm birth and even death of the fetus or mother. The frequency is 5-8% of all pregnancies but can be much greater in certain groups, e.g. women carrying twins. Preeclampsia accounts for 25% of maternal deaths in the United States (Winn V D et al. Reprod Sci 2007; 14: 508-23; Shah D M. Curr Ob in Nephrol Hypertens 2007; 16:213-20).

Early-onset and late-onset preeclampsia are now considered to be two separate disorders (Crispi F., et al. Ultrasound Obstet Gynecol 2008; 31:303-9; Eggier M et al BJOG; 113 (5): 580-9; Redman C W et al. Science 2005; 308:1592-4). Preeclampsia is considered to be early-onset if it occurs starting from the 20th week to 34th week of gestation and requires delivery prior to 34 weeks, and late-onset if it starts on or after 34 weeks of gestation. Early-onset preeclampsia is associated with greater rates of maternal complications (morbidities), long-term problems, and complications in the fetus. On the other hand, pregnancies with late-onset preeclampsia have similar rates of maternal and newborn complications as pregnancies with normal blood pressure (Crispi F., et al. Ultrasound Obstet Gynecol 2008; 31: 303-9; Sibai B M et al. Seminars Prenatal 2003; 27: 239-46). This may be a result of the two conditions having different mechanisms of action as previously suggested (see, e.g., Sibai B et al. Lancet 2005; 365:785-99; Vattern L J et al BJOG 2004; 111: 298-302; Crispi F, et al. Ultrasound Obstet Gynecol 2008; 31:303-9).

Early-onset preeclampsia is thought to result from a profound failure of development of the placental tissues (“trophoblast”), while late-onset preeclampsia occurs in the presence of a normal or only mildly affected placenta. Since the placenta is responsible for the nourishment and transfer of oxygen to the fetus, early-onset preeclampsia is not surprisingly associated with greater risk to the fetus and newborn. In late-onset preeclampsia, the placental dysfunction or failure of the trophoblast development is thought to be mild or non-existent. The latter condition is primarily due to preexisting disorders of maternal blood vessels or preexisting maternal hypertension, diabetes or metabolic abnormalities (Wilkstrom A K et al., Obstet General 2007; 109:1368-74; McElrath T F et al., Am J Epidemiol 2008; 168:980-9) interacting with a mildly deficient placental development.

Extensive efforts have been made to develop markers that can accurately predict preeclampsia. Biochemical markers and Doppler ultrasound measurements of blood flow in the maternal uterine arteries have been tested extensively but almost none of these have thus far achieved widespread clinical use (Conde-Agudelo A., et al., Obstet General 2004; 104:1367-91). Markers for preeclampsia prediction that have been evaluated in the past include placental growth factor (PLGF), inhibin A, tumor necrosis factor receptor-1 (TNF-R1), PP-13 (placental protein-13) and PAPP-A (placental associated plasma protein-A), combined with uterine artery Doppler flow measurements and maternal demographic characteristics such as race/ethnicity and body weight (Poon L. C., et al., Ultrasound Obstet General 2010; 35: 662-70; Akolekar R et al. Prenat Diag 2009; 29: 1103-8).

There remains a need to develop reliable and clinically useful markers for predicting preeclampsia. Being able to identify pregnant women at risk for developing early-onset or late-onset preeclampsia could permit the use of prophylactic agents, such as aspirin which have been shown to be effective for prevention of preeclampsia development if applied early enough, i.e., in the first trimester.

### SUMMARY OF THE INVENTION

It is one object of the present invention to provide a method for predicting a pregnant woman's risk for developing early-onset preeclampsia by a) measuring concentrations of one or more metabolites selected from the group consisting of acetate, alanine, arginine, choline, creatine, dimethylamine, acetamide, trimethylamine, glutamine, citrate, ethanol, formate, glycerol, glycine, leucine, methanol, methionine, ornithine, phenylalanine, propylene glycol, serine, succinate, hydroxy-isovalerate, pyruvate, hydroxy-buturate and threonine in the pregnant woman's bodily fluid; b) comparing the pregnant woman's one or more metabolite concentrations to concentrations of corresponding one or more metabolites obtained from pregnant women with early-onset preeclampsia and to concentrations of the corresponding one or more metabolites obtained from pregnant women exhibiting normal blood pressure and normal protein levels in urine, wherein all metabolite concentrations are measured at same or similar gestational age; and c) predicting the pregnant woman's risk of developing early-onset preeclampsia, wherein a statistically significant change in the concentration of the one or more metabolites between the pregnant woman and the corresponding one or more metabolites from the pregnant women exhibiting normal blood pressure and normal protein levels in urine indicates a greater probability of developing early-onset preeclampsia.

Another object of the present invention is directed to a method for predicting a pregnant woman's risk for developing early-onset preeclampsia, the method comprising measuring concentrations of one or more metabolites selected from the group consisting of acetate, alanine, arginine, choline, creatine, dimethylamine, acetamide, trimethylamine, glutamine, citrate, ethanol, formate, glycerol, glycine, leucine, methanol, methionine, ornithine, phenylalanine, propylene glycol, serine, succinate, hydroxy-isovalerate, pyruvate, hydroxy-buturate and threonine in the pregnant woman's bodily fluid; measuring crown rump length (CRL) of the pregnant woman's fetus; comparing the pregnant woman's one or more metabolite concentrations to concentrations of corresponding one or more metabolites obtained from pregnant women with early-onset preeclampsia and to concentrations of the corresponding one or more metabolites obtained from pregnant women exhibiting normal blood pressure and normal protein levels in urine, wherein all metabolite concentrations are standardized according to CRL average values from pregnant women with early-onset preeclampsia and pregnant women exhibiting normal blood pressure and normal protein levels in urine; and predicting the pregnant woman's risk of developing early-onset preeclampsia, wherein a statistically significant change in the concentration of the one or more metabolites between the pregnant woman and the corresponding one or more standardized metabolites from the pregnant women exhibiting normal blood pressure and normal protein levels in urine indicates a greater probability of developing early-onset preeclampsia.

The present invention is also directed to a method for predicting a pregnant woman's risk for developing late-onset preeclampsia by a) measuring concentrations of one or more metabolites selected from the group consisting of 2-hydroxy-butyrate, acetamide, acetate, acetone, carnitine, creatine, creatinine, dimethylamine, glucose, glycerol, propylene glycol, ethylene glycol, threonine, alanine, trimethylamine, 3-hydroxy-butyrate, valine, pyruvate and methylhistidine in the pregnant woman's bodily fluid; b) comparing the pregnant woman's one or more metabolite concentrations to the corresponding one or more metabolite concentrations obtained from pregnant women with late-onset preeclampsia and pregnant women exhibiting normal blood pressure and normal protein levels in urine, wherein all metabolite concentrations are measured at the same or similar gestational age; and c) predicting the pregnant woman's risk of developing late-onset preeclampsia, wherein the statistically significant change in concentration of one or more metabolites between the pregnant woman and the corresponding one or more metabolites from the pregnant women exhibiting normal blood pressure and normal protein levels in urine indicates a greater probability of developing late-onset preeclampsia.

Another object of the present invention is the provision of a method for predicting a pregnant woman's risk for developing late-onset preeclampsia, the method comprising measuring concentrations of one or more metabolites selected from the group consisting of 2-hydroxy-butyrate, acetamide, acetate, acetone, carnitine, creatine, creatinine, dimethylamine, glucose, glycerol, propylene glycol, ethylene glycol, threonine, alanine, trimethylamine, 3-hydroxy-butyrate, valine, pyruvate and methylhistidine in the pregnant woman's bodily fluid; measuring crown rump length (CRL) of the pregnant woman's fetus; comparing the pregnant woman's one or more metabolite concentrations to the corresponding one or more metabolite concentrations obtained from pregnant women with late-onset preeclampsia and pregnant women exhibiting normal blood pressure and normal protein levels in urine, wherein all metabolite concentrations are standardized according to CRL average values of fetuses from pregnant women with late-onset preeclampsia and pregnant women exhibiting normal blood pressure and normal protein levels in urine; and predicting the pregnant woman's risk of developing late-onset preeclampsia, wherein the statistically significant change in concentration of one or more metabolites between the pregnant woman and the corresponding one or more standardized metabolites from the pregnant women exhibiting normal blood pressure and normal protein levels in urine indicates a greater probability of developing late-onset preeclampsia.

The present invention also relates to a computer-readable medium having stored thereon an array of normalized metabolite concentration values and a program of instructions executable by a processor to compare a pregnant woman's bodily fluid sample metabolite concentration value to a corresponding normalized metabolite concentration value obtained from pregnant women exhibiting normal blood pressure and normal protein levels in urine to predict the pregnant woman's risk for developing early-onset preeclampsia or late-onset preeclampsia.

Other objects and features will be in part apparent and in part pointed out hereinafter.

### BRIEF DESCRIPTION OF THE DRAWINGS

FIG. 1 shows the partial least squares discriminant analysis plot (PLS-DA) indicating the separation (distinction) between early-onset preeclampsia (in green) and normal control cases (in red) achieved by NMR analysis as described in Examples 1-3. It shows the separation achieved by 3 components (metabolites) used as discriminators and their degree (%) of individual contribution to the overall separation of the two groups. The contribution of each is denoted on the x-, y-, and z-axes. Permutation testing using 2000 random sampling of the data indicated that the separation of the two groups was statistically significant, p=0.01.

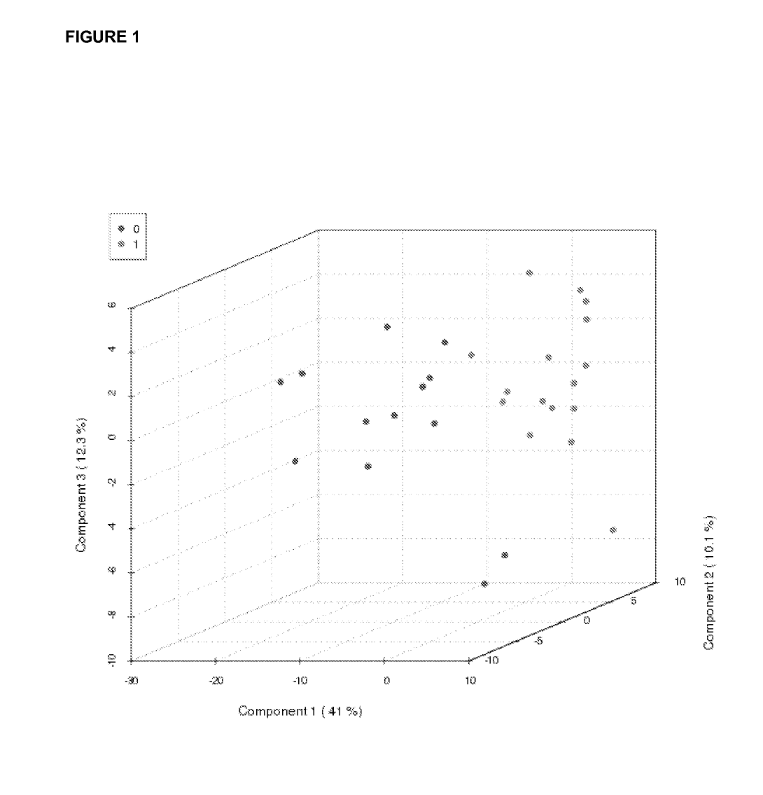

FIG. 2 shows the principal component analysis (PCA) plot indicating the separation achieved between late-onset preeclampsia (green) and normal (red) cases based on NMR analysis as described in Examples 1-3. The principal components (i.e. two main metabolite sets) primarily responsible for distinguishing the groups are shown on the x-, y-axes. Permutation testing based on 2000 random sampling of the data indicated that the separation of the two groups was statistically significant, p=0.02.

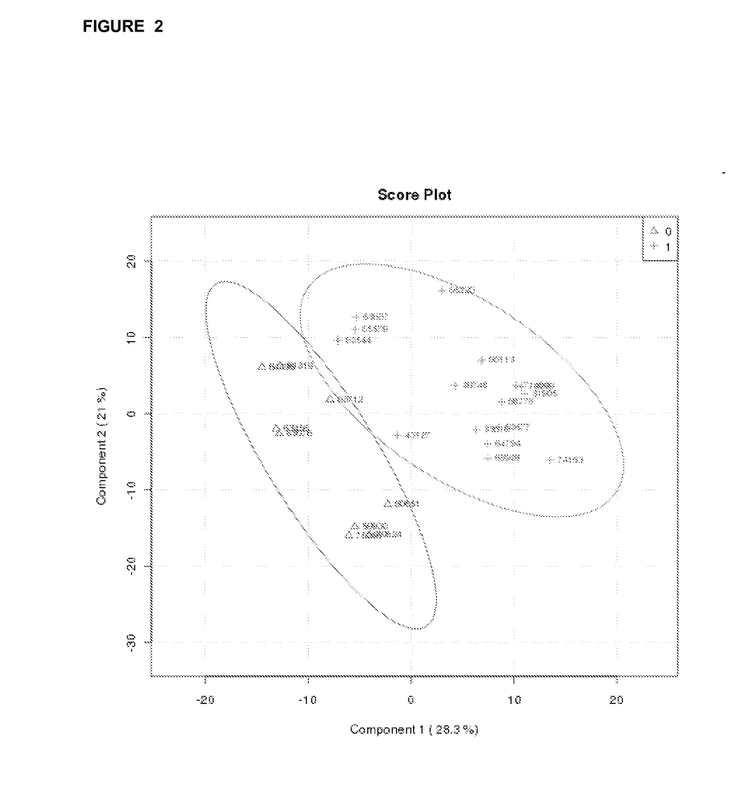

FIG. 3 shows the partial least squares-discriminant analysis (PLS-DA) plot demonstrating the separation achieved between early-onset (green) and late-onset (red) preeclampsia cases using NMR as described in Examples 1-3. The percentage contribution of the two main metabolites to the separation of the early-onset and normal patient groups are shown in the plot on the x-, and y-axis. Permutation analysis using 2000 random sampling and relabeling of the dataset indicated that the separation of the two groups achieved was statistically significant, p=0.02.

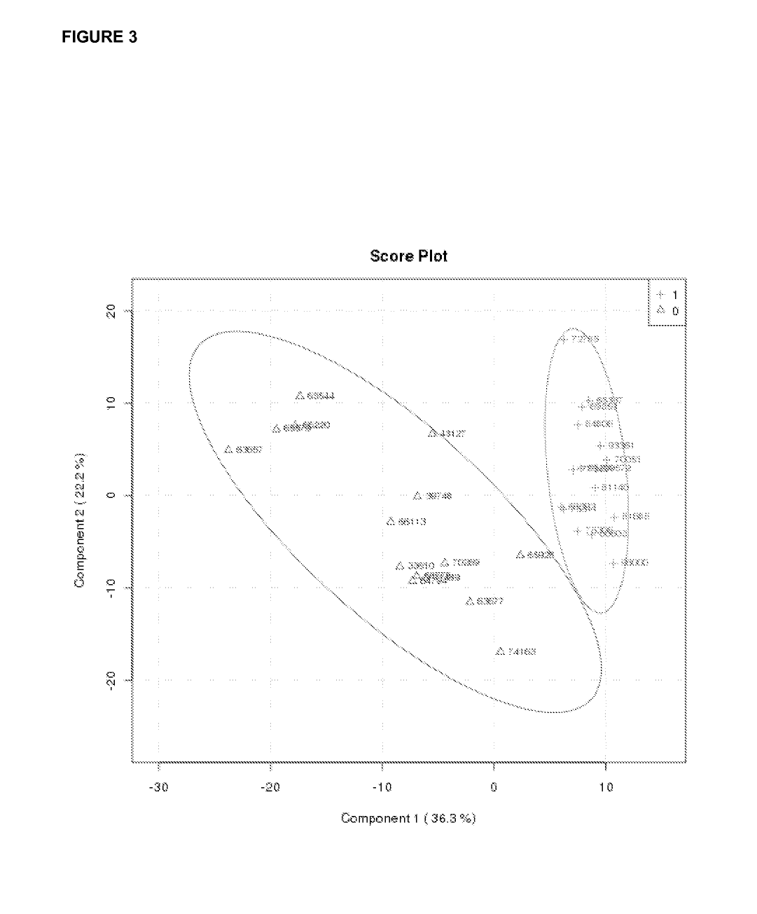

FIG. 4 shows a principal component analysis plot showing the separation between early PE (in green) and control (in red) for nuclear magnetic resonance spectrometry as described in Example 4.

FIG. 5 depicts separation between the cases of early preeclampsia (in green) and controls (in red) in the partial least squares discriminant analysis two dimensional score plot (a) and three dimensional score plot (b) as described in Example 4.

FIG. 6 depicts a variable importance in projection (VIP) plot indicating the most discriminating metabolites in descending order of importance as described in Example 4.

FIG. 7 shows an ROC curve for prediction of early-onset preeclampsia based on a metabolite-only algorithm as described in Example 4.

FIG. 8 depicts an ROC curve for prediction of early-onset preeclampsia based on a metabolite plus uterine artery Doppler algorithm as described in Example 4.

FIG. 9a depicts Principal Components Analysis showing the separation between late PE (in green) and control (in red) for NMR as described in Example 5. Clustering and segregation of the two patient groups indicate that significant discrimination of groups were achieved based on metabolite concentration differences. FIG. 9b depicts Partial Least Squares Discriminant Analysis wherein late onset preeclampsia cases are indicated in green and controls in red. 2000 permutations or resamplings were performed (p<0.0005).

FIG. 10 shows a variable importance in projection (VIP) plot as described in Example 5. The most discriminating metabolites are shown in descending order of importance. The color boxes indicate whether metabolite concentration is increased or decreased.

FIG. 11 shows an ROC curve for the prediction of late-onset preeclampsia as described in Example 5. (Area under ROC curve (95% CI), 0.908 (0.839, 0.977), p<0.001). Markers that were used are maternal race, glycerol, trimethylamine, valine and methylhistidine.

### DEFINITIONS AND ABBREVIATIONS

As used herein, the terms “one or more” and “at least one” in the context of biomarkers, such as metabolites mean any one, two, three, four, etc. of the listed members within a group, in any permutation. Accordingly, the terms “one or more” and “at least one” include any two, any three, any four, etc. of the members specifically listed within a group. Thus, the invention is not limited to any single group or subset of biomarkers. It is emphasized that the terms “one or more” and “at least one” are used in the broadest sense, and are used to designate any subgroup within a group with multiple members. Similarly, the terms “at least 2,” “at least 3,” “at least 4,” etc., cover any combinations of the members within a particular group, provided that the total number of members within the combination is at least 2, at least 3, at least 4, etc.

The term “ionization” and “ionizing” as used herein refers to the process of generating an analyte ion having a net electrical charge equal to one or more electron units. Negative ions are those ions having a net negative charge of one or more electron units, while positive ions are those ions having a net positive charge of one or more electron units.

The term “desorption” as used herein refers to the removal of an analyte from a surface and/or the entry of an analyte into a gaseous phase.

The terms “mass spectrometry” or “MS” as used herein refer to methods of filtering, detecting, and measuring ions based on their mass-to-charge ratio, or “m/z.”

The term “matrix-assisted laser desorption ionization,” or “MALDI” as used herein refers to methods in which a non-volatile sample is exposed to laser irradiation, which desorbs and ionizes analytes in the sample by various ionization pathways, including photo-ionization, protonation, deprotonation, and cluster decay.

As used herein, the definition of preeclampsia is based on the International Society for the Study of Hypertension in Pregnancy (Brown M A et al., The classification and diagnosis of hypertensive disorders of pregnancy: Statement from the International Society of Hypertension in Pregnancy (ISSHP). Hypertension Pregnancy 2001; 20: ix-xv). Based on this definition, systolic blood pressure should be 140 mmHg or more and/or the diastolic blood pressure should be 90 mmHg or more on at least two occasions 4 hours apart and developing after 20 weeks gestation in a previously normotensive woman. In addition, there should be a significant amount of protein in the urine (proteinuria) defined as 300 mg in the total volume of urine collected over a 24 hour period. Alternatively, significant proteinuria was defined as at least 2+ based on a semi-quantitative measurement using urine dipstick from a specimen of mid-stream urine or a catheter urine specimen if a 24 hour urine collection is not available. “Early-onset” preeclampsia refers to preeclampsia developing and requiring delivery prior to the 34th week of gestation, and “late-onset” preeclampsia refers to preeclampsia developing during or after 34 weeks of gestation.

### DESCRIPTION OF THE PREFERRED EMBODIMENTS

The present invention is directed to methods for predicting a pregnant woman's risk of developing early-onset or late-onset preeclampsia. The methods are based on measuring one or more metabolites obtained from a pregnant woman's bodily fluid, such as blood or urine and comparing the concentration of one or more of these metabolites to the corresponding ones isolated from pregnant women with early-onset or late-onset preeclampsia (i.e., normalized metabolite concentration(s) from pregnant women with early-onset or late-onset preeclampsia) and pregnant women exhibiting normal blood pressure and normal protein levels in urine (i.e., normalized metabolite concentration(s) from normal control pregnant women).

A method for predicting a pregnant woman's risk for developing early-onset preeclampsia comprises measuring concentrations of one or more metabolites selected from the group consisting of acetate, alanine, arginine, choline, creatine, dimethylamine, acetamide, trimethylamine, glutamine, citrate, ethanol, formate, glycerol, glycine, leucine, methanol, methionine, ornithine, phenylalanine, propylene glycol, serine, succinate, hydroxy-isovalerate, pyruvate, hydroxy-buturate and threonine in the pregnant woman's bodily fluid. The pregnant woman's one or more metabolite concentrations are compared to the corresponding one or more metabolite concentrations obtained from pregnant women with early-onset preeclampsia and to the corresponding one or more metabolite concentrations obtained from pregnant women exhibiting normal blood pressure and normal protein levels in urine. All metabolite concentrations are measured at the same or similar gestational age. The pregnant woman's risk of developing early-onset preeclampsia is predicted, wherein the statistically significant change in concentration of one or more metabolites between the pregnant woman and the corresponding one or more metabolites from the pregnant women exhibiting normal blood pressure and normal protein levels in urine indicates a greater probability of developing early-onset preeclampsia.

Additionally, metabolite concentrations obtained from pregnant women with early-onset preeclampsia and from pregnant women exhibiting normal blood pressure and normal protein levels in urine can be standardized according to fetal crown-rump-length (CRL) average values prior to being compared to corresponding metabolite concentrations of a pregnant woman being tested.

A method for determining a pregnant woman's risk for developing late-onset preeclampsia comprises measuring concentrations of one or more metabolites selected from the group consisting of 2-hydroxy-butyrate, acetamide, acetate, acetone, carnitine, creatine, creatinine, dimethylamine, glucose, glycerol, propylene glycol, ethylene glycol, threonine, alanine, trimethylamine, 3-hydroxy-butyrate, valine, pyruvate and methylhistidine in the pregnant woman's bodily fluid. The pregnant woman's one or more metabolite concentrations are compared to the corresponding one or more metabolite concentrations obtained from pregnant women with late-onset preeclampsia and to the corresponding one or more metabolite concentrations obtained from pregnant women exhibiting normal blood pressure and normal protein levels in urine. All metabolite concentrations are measured at the same or similar gestational age. The pregnant woman's risk of developing late-onset preeclampsia is predicted, wherein the statistically significant change in concentration of one or more metabolites between the pregnant woman and the corresponding one or more metabolites from the pregnant women exhibiting normal blood pressure and normal protein levels in urine indicates a greater probability of developing late-onset preeclampsia.

Similarly as for early preeclampsia described above, metabolite concentrations obtained from pregnant women with late-onset preeclampsia and from pregnant women exhibiting normal blood pressure and normal protein levels in urine can be standardized according to fetal crown-rump-length (CRL) average values prior to being compared to corresponding metabolite concentrations of a pregnant woman being tested.

Measuring concentrations of one or more metabolites is performed by obtaining the pregnant woman's bodily fluid containing one or more of these metabolites. The bodily fluid can be blood, such as a dried blood sample, a blood serum sample or a blood plasma sample. The bodily fluid can also be urine. Other suitable maternal bodily fluids for use in the methods of the invention include, for example, amniotic fluid, cerebrospinal fluid, mucus, and saliva.

Preferably, a bodily sample such as blood or urine is obtained from a pregnant woman during the first trimester of pregnancy, such as at a gestational age from 10 weeks to 18 weeks, from 11 weeks to 14 weeks, or from 11 weeks to 13 weeks. Early-onset preeclampsia develops from 20 weeks to 34 weeks of gestation, and late-onset preeclampsia develops from 34 weeks of gestation until delivery.

One or more metabolites used to predict a pregnant woman's risk of developing early-onset preeclampsia are selected from the group consisting of acetate, alanine, arginine, choline, creatine, dimethylamine, acetamide, trimethylamine, glutamine, citrate, ethanol, formate, glycerol, glycine, leucine, methanol, methionine, ornithine, phenylalanine, propylene glycol, serine, succinate, hydroxy-isovalerate, pyruvate, hydroxy-buturate and threonine. For example, one or more metabolites used to predict early-onset preeclampsia are selected from acetate, choline, creatine, dimethylamine, acetamide, trimethylamine, glutamine, citrate, ethanol, formate, glycerol, glycine, leucine, methanol, methionine, ornithine, propylene glycol, serine, succinate, hydroxy-isovalerate, pyruvate, hydroxy-buturate and threonine. In additional examples, one or more metabolites for predicting early-onset preeclampsia are selected from acetate, dimethylamine, acetamide, succinate, trimethylamine, glutamine, citrate, and ornithine. In other embodiments, one or more metabolites are selected from 1) creatine and choline, 2) propylene glycol and formate, 3) citrate, glycerol, hydroxy-isovalerate and methionine, 4) acetate, glutamine, pyruvate, propylene glycol, trimethylamine and hydroxy-buturate, or 5) pyruvate, propylene glycol, trimethylamine and hydroxy-isovalerate.

Creatine can be a single metabolite used to predict a pregnant woman's risk of developing early-onset preeclampsia. It will be obvious to a skilled artisan that many different combinations of the above-mentioned metabolites for early-onset preeclampsia can be tested, which include different combinations of 2, 3, 4, 5, 6, 7, 8, 9, 10, 11, 12, 13, 14, 15, 16, 17, 18, 19, 20, 21, 22, 23, 24 and 25 above-mentioned metabolites. Single metabolites and the combination of all 23 metabolites can also be used to test for early-onset preeclampsia.

One or more metabolites used to predict a pregnant woman's risk of developing late-onset preeclampsia are selected from the group consisting of 2-hydroxy-butyrate, acetamide, acetate, acetone, carnitine, creatine, creatinine, dimethylamine, glucose, glycerol, propylene glycol, ethylene glycol, threonine, alanine, trimethylamine, 3-hydroxy-butyrate, valine, pyruvate and methylhistidine. Metabolites used to predict late-onset preeclampsia can also be selected from 2-hydroxy-butyrate, acetamide, acetate, acetone, carnitine, creatine, creatinine, dimethylamine, glucose, glycerol, propylene glycol, ethylene glycol, threonine, trimethylamine, 3-hydroxy-butyrate, valine, pyruvate and methylhistidine. For example, the one or more metabolites for predicting late-onset preeclampsia can be selected from glycerol, choline and alanine. In other embodiments, one or more metabolites are selected from 1) glycerol, ethylene glycol, threonine, carnitine, and alanine, 2) 3-hydroxy-butyrate, glycerol, trimethylamine, valine and methylhistidine or 3) glycerol, pyruvate, trimethylamine, valine and methylhistidine.

Also, glycerol can be a single metabolite used to predict a pregnant woman's risk of developing late-onset preeclampsia. It will be obvious to a skilled artisan that many different combinations of the above-mentioned metabolites for late-onset preeclampsia can be tested, which include different combinations of 2, 3, 4, 5, 6, 7, 8, 9, 10, 11, 12, 13, 14, 15, 16, 17 and 18 above-mentioned metabolites. Single metabolites and the combination of all 16 metabolites can also be used to test for late-onset preeclampsia.

Representative combinations of 2 metabolites used to predict a pregnant woman's risk of developing early-onset preeclampsia include:

| 2 Metabolite Combinations | | 2 Metabolite Combinations | |
|---|---|---|---|
| acetate | alanine | alanine | Arginine |
| acetate | arginine | alanine | Choline |
| acetate | choline | alanine | creatine |
| acetate | creatine | alanine | dimethylamine |
| acetate | dimethylamine | alanine | acetamide |
| acetate | acetamide | alanine | trimethylamine |
| acetate | trimethylamine | alanine | glutamine |
| acetate | glutamine | alanine | citrate |
| acetate | citrate | alanine | ethanol |
| acetate | ethanol | alanine | formate |
| acetate | formate | alanine | glycerol |
| acetate | glycerol | alanine | glycine |
| acetate | glycine | alanine | leucine |
| acetate | leucine | alanine | methanol |
| acetate | methanol | alanine | methionine |
| acetate | methionine | alanine | ornithine |
| acetate | ornithine | alanine | phenylalanine |
| acetate | phenylalanine | alanine | propylene glycol |
| acetate | propylene glycol | alanine | serine |
| acetate | serine | alanine | succinate |
| acetate | succinate | alanine | threonine |
| acetate | threonine | arginine | choline |
| choline | creatine | arginine | creatine |
| choline | dimethylamine | arginine | dimethylamine |
| choline | acetamide | arginine | acetamide |
| choline | trimethylamine | arginine | trimethylamine |
| choline | glutamine | arginine | glutamine |
| choline | citrate | arginine | citrate |
| choline | ethanol | arginine | ethanol |
| choline | formate | arginine | formate |
| choline | glycerol | arginine | glycerol |
| choline | glycine | arginine | glycine |
| choline | leucine | arginine | leucine |
| choline | methanol | arginine | methanol |
| choline | methionine | arginine | methionine |
| choline | ornithine | arginine | ornithine |
| choline | phenylalanine | arginine | phenylalanine |
| choline | propylene glycol | arginine | propylene glycol |
| choline | serine | arginine | serine |
| choline | succinate | arginine | succinate |
| choline | threonine | arginine | threonine |
| creatine | dimethylamine | dimethylamine | acetamide |
| creatine | acetamide | dimethylamine | trimethylamine |
| creatine | trimethylamine | dimethylamine | glutamine |
| creatine | glutamine | dimethylamine | citrate |
| creatine | citrate | dimethylamine | ethanol |
| creatine | ethanol | dimethylamine | formate |
| creatine | formate | dimethylamine | glycerol |
| creatine | glycerol | dimethylamine | glycine |
| creatine | glycine | dimethylamine | leucine |
| creatine | leucine | dimethylamine | methanol |
| creatine | methanol | dimethylamine | methionine |
| creatine | methionine | dimethylamine | ornithine |
| creatine | ornithine | dimethylamine | phenylalanine |
| creatine | phenylalanine | dimethylamine | propylene glycol |
| creatine | propylene glycol | dimethylamine | serine |
| creatine | serine | dimethylamine | succinate |
| creatine | succinate | dimethylamine | threonine |
| creatine | threonine | acetamide | trimethylamine |
| trimethylamine | glutamine | acetamide | glutamine |
| trimethylamine | citrate | acetamide | citrate |
| trimethylamine | ethanol | acetamide | ethanol |
| trimethylamine | formate | acetamide | formate |
| trimethylamine | glycerol | acetamide | glycerol |
| trimethylamine | glycine | acetamide | glycine |
| trimethylamine | leucine | acetamide | leucine |
| trimethylamine | methanol | acetamide | methanol |
| trimethylamine | methionine | acetamide | methionine |
| trimethylamine | ornithine | acetamide | ornithine |
| trimethylamine | phenylalanine | acetamide | phenylalanine |
| trimethylamine | propylene glycol | acetamide | propylene glycol |
| trimethylamine | serine | acetamide | serine |
| trimethylamine | succinate | acetamide | succinate |
| trimethylamine | threonine | acetamide | threonine |
| glutamine | citrate | citrate | ethanol |
| glutamine | ethanol | citrate | formate |
| glutamine | formate | citrate | glycerol |
| glutamine | glycerol | citrate | glycine |
| glutamine | glycine | citrate | leucine |
| glutamine | leucine | citrate | methanol |
| glutamine | methanol | citrate | methionine |
| glutamine | methionine | citrate | ornithine |
| glutamine | ornithine | citrate | phenylalanine |
| glutamine | phenylalanine | citrate | propylene glycol |
| glutamine | propylene glycol | citrate | serine |
| glutamine | serine | citrate | succinate |
| glutamine | succinate | citrate | threonine |
| glutamine | threonine | ethanol | formate |
| formate | glycerol | ethanol | glycerol |
| formate | glycine | ethanol | glycine |
| formate | leucine | ethanol | leucine |
| formate | methanol | ethanol | methanol |
| formate | methionine | ethanol | methionine |
| formate | ornithine | ethanol | ornithine |
| formate | phenylalanine | ethanol | phenylalanine |
| formate | propylene glycol | ethanol | propylene glycol |
| formate | serine | ethanol | serine |
| formate | succinate | ethanol | succinate |
| formate | threonine | ethanol | threonine |
| Glycerol | glycine | glycine | leucine |
| glycerol | leucine | glycine | methanol |
| glycerol | methanol | glycine | methionine |
| glycerol | methionine | glycine | ornithine |
| glycerol | ornithine | glycine | phenylalanine |
| glycerol | phenylalanine | glycine | propylene glycol |
| glycerol | propylene glycol | glycine | serine |
| glycerol | serine | glycine | succinate |
| glycerol | succinate | glycine | threonine |
| glycerol | threonine | leucine | methanol |
| methanol | methionine | leucine | methionine |
| methanol | ornithine | leucine | ornithine |
| methanol | phenylalanine | leucine | phenylalanine |
| methanol | propylene glycol | leucine | propylene glycol |
| methanol | serine | leucine | serine |
| methanol | succinate | leucine | succinate |
| methanol | threonine | leucine | threonine |
| methionine | ornithine | ornithine | phenylalanine |
| methionine | phenylalanine | ornithine | propylene glycol |
| methionine | propylene glycol | ornithine | serine |
| methionine | serine | ornithine | succinate |
| methionine | succinate | ornithine | threonine |
| methionine | threonine | phenylalanine | propylene glycol |
| propylene glycol | serine | phenylalanine | serine |
| propylene glycol | succinate | phenylalanine | succinate |
| propylene glycol | threonine | phenylalanine | threonine |
| serine | succinate | Succinate | threonine |
| serine | threonine | | |

Representative examples of combinations of 3 metabolites used to predict a pregnant woman's risk of developing early-onset preeclampsia include:

| 3 Metabolite Combinations | | | 3 Metabolite Combinations | | |
|---|---|---|---|---|---|
| acetate | alanine | arginine | alanine | arginine | choline |
| acetate | alanine | choline | alanine | arginine | creatine |
| acetate | alanine | creatine | alanine | arginine | dimethylamine |
| acetate | alanine | dimethylamine | alanine | arginine | acetamide |
| acetate | alanine | acetamide | alanine | arginine | trimethylamine |
| acetate | alanine | trimethylamine | alanine | arginine | glutamine |
| acetate | alanine | glutamine | alanine | arginine | citrate |
| acetate | alanine | citrate | alanine | arginine | ethanol |
| acetate | alanine | ethanol | alanine | arginine | formate |
| acetate | alanine | formate | alanine | arginine | glycerol |
| acetate | alanine | glycerol | alanine | arginine | glycine |
| acetate | alanine | glycine | alanine | arginine | leucine |
| acetate | alanine | leucine | alanine | arginine | methanol |
| acetate | alanine | methanol | alanine | arginine | methionine |
| acetate | alanine | methionine | alanine | arginine | ornithine |
| acetate | alanine | ornithine | alanine | arginine | phenylalanine |
| acetate | alanine | phenylalanine | alanine | arginine | propylene glycol |
| acetate | alanine | propylene glycol | alanine | arginine | serine |
| acetate | alanine | serine | alanine | arginine | succinate |
| acetate | alanine | succinate | alanine | arginine | threonine |
| acetate | alanine | threonine | arginine | choline | creatine |
| choline | creatine | dimethylamine | arginine | choline | dimethylamine |
| choline | creatine | acetamide | arginine | choline | acetamide |
| choline | creatine | trimethylamine | arginine | choline | trimethylamine |
| choline | creatine | glutamine | arginine | choline | glutamine |
| choline | creatine | citrate | arginine | choline | citrate |
| choline | creatine | ethanol | arginine | choline | ethanol |
| choline | creatine | formate | arginine | choline | formate |
| choline | creatine | glycerol | arginine | choline | glycerol |
| choline | creatine | glycine | arginine | choline | glycine |
| choline | creatine | leucine | arginine | choline | leucine |
| choline | creatine | methanol | arginine | choline | methanol |
| choline | creatine | methionine | arginine | choline | methionine |
| choline | creatine | ornithine | arginine | choline | ornithine |
| choline | creatine | phenylalanine | arginine | choline | phenylalanine |
| choline | creatine | propylene glycol | arginine | choline | propylene glycol |
| choline | creatine | serine | arginine | choline | serine |
| choline | creatine | succinate | arginine | choline | succinate |
| choline | creatine | threonine | arginine | choline | threonine |
| creatine | dimethylamine | acetamide | dimethylamine | acetamide | trimethylamine |
| creatine | dimethylamine | trimethylamine | dimethylamine | acetamide | glutamine |
| creatine | dimethylamine | glutamine | dimethylamine | acetamide | citrate |
| creatine | dimethylamine | citrate | dimethylamine | acetamide | ethanol |
| creatine | dimethylamine | ethanol | dimethylamine | acetamide | formate |
| creatine | dimethylamine | formate | dimethylamine | acetamide | glycerol |
| creatine | dimethylamine | glycerol | dimethylamine | acetamide | glycine |
| creatine | dimethylamine | glycine | dimethylamine | acetamide | leucine |
| creatine | dimethylamine | leucine | dimethylamine | acetamide | methanol |
| creatine | dimethylamine | methanol | dimethylamine | acetamide | methionine |
| creatine | dimethylamine | methionine | dimethylamine | acetamide | ornithine |
| creatine | dimethylamine | ornithine | dimethylamine | acetamide | phenylalanine |
| creatine | dimethylamine | phenylalanine | dimethylamine | acetamide | propylene glycol |
| creatine | dimethylamine | propylene glycol | dimethylamine | acetamide | serine |
| creatine | dimethylamine | serine | dimethylamine | acetamide | succinate |
| creatine | dimethylamine | succinate | dimethylamine | acetamide | threonine |
| creatine | dimethylamine | threonine | acetamide | glutamine | citrate |
| glutamine | ethanol | formate | acetamide | glutamine | ethanol |
| glutamine | ethanol | glycerol | acetamide | glutamine | formate |
| glutamine | ethanol | glycine | acetamide | glutamine | glycerol |
| glutamine | ethanol | leucine | acetamide | glutamine | glycine |
| glutamine | ethanol | methanol | acetamide | glutamine | leucine |
| glutamine | ethanol | methionine | acetamide | glutamine | methanol |
| glutamine | ethanol | ornithine | acetamide | glutamine | methionine |
| glutamine | ethanol | phenylalanine | acetamide | glutamine | ornithine |
| glutamine | ethanol | propylene glycol | acetamide | glutamine | phenylalanine |
| glutamine | ethanol | serine | acetamide | glutamine | propylene glycol |
| glutamine | ethanol | succinate | acetamide | glutamine | serine |
| glutamine | ethanol | threonine | acetamide | glutamine | succinate |
| ethanol | formate | glycerol | acetamide | glutamine | threonine |
| ethanol | formate | glycine | formate | glycerol | glycine |
| ethanol | formate | leucine | formate | glycerol | leucine |
| ethanol | formate | methanol | formate | glycerol | methanol |
| ethanol | formate | methionine | formate | glycerol | methionine |
| ethanol | formate | ornithine | formate | glycerol | ornithine |
| ethanol | formate | phenylalanine | formate | glycerol | phenylalanine |
| ethanol | formate | propylene glycol | formate | glycerol | propylene glycol |
| ethanol | formate | serine | formate | glycerol | serine |
| ethanol | formate | succinate | formate | glycerol | succinate |
| ethanol | formate | threonine | formate | glycerol | threonine |
| glycerol | glycine | leucine | glycine | leucine | methanol |
| glycerol | glycine | methanol | glycine | leucine | methionine |
| glycerol | glycine | methionine | glycine | leucine | ornithine |
| glycerol | glycine | ornithine | glycine | leucine | phenylalanine |
| glycerol | glycine | phenylalanine | glycine | leucine | propylene glycol |
| glycerol | glycine | propylene glycol | glycine | leucine | serine |
| glycerol | glycine | serine | glycine | leucine | succinate |
| glycerol | glycine | succinate | glycine | leucine | threonine |
| glycerol | glycine | threonine | leucine | methanol | methionine |
| methanol | methionine | ornithine | leucine | methanol | ornithine |
| methanol | methionine | phenylalanine | leucine | methanol | phenylalanine |
| methanol | methionine | propylene glycol | leucine | methanol | propylene glycol |
| methanol | methionine | serine | leucine | methanol | serine |
| methanol | methionine | succinate | leucine | methanol | succinate |
| methanol | methionine | threonine | leucine | methanol | threonine |
| methionine | ornithine | phenylalanine | ornithine | phenylalanine | propylene glycol |
| methionine | ornithine | propylene glycol | ornithine | phenylalanine | serine |
| methionine | ornithine | serine | ornithine | phenylalanine | succinate |
| methionine | ornithine | succinate | ornithine | phenylalanine | threonine |
| methionine | ornithine | threonine | phenylalanine | propylene glycol | serine |
| propylene glycol | serine | succinate | phenylalanine | propylene glycol | succinate |
| propylene glycol | serine | threonine | phenylalanine | propylene glycol | threonine |
| Serine | Succinate | threonine | | | |

Representative combinations of 4 metabolites used to predict a pregnant woman's risk of developing early-onset preeclampsia include:

4 Metabolite Combinations

| | | | |
|---|---|---|---|
| acetate | alanine | arginine | choline |
| acetate | alanine | arginine | creatine |
| acetate | alanine | arginine | dimethylamine |
| acetate | alanine | arginine | acetamide |
| acetate | alanine | arginine | trimethylamine |
| acetate | alanine | arginine | glutamine |
| acetate | alanine | arginine | citrate |
| acetate | alanine | arginine | ethanol |
| acetate | alanine | arginine | formate |
| acetate | alanine | arginine | glycerol |
| acetate | alanine | arginine | glycine |
| acetate | alanine | arginine | leucine |
| acetate | alanine | arginine | methanol |
| acetate | alanine | arginine | methionine |
| acetate | alanine | arginine | ornithine |
| acetate | alanine | arginine | phenylalanine |
| acetate | alanine | arginine | propylene |
| | | | glycol |
| acetate | alanine | arginine | serine |
| acetate | alanine | arginine | succinate |
| acetate | alanine | arginine | threonine |
| alanine | arginine | choline | creatine |
| alanine | arginine | choline | dimethylamine |
| alanine | arginine | choline | acetamide |
| alanine | arginine | choline | trimethylamine |
| alanine | arginine | choline | glutamine |
| alanine | arginine | choline | citrate |
| alanine | arginine | choline | ethanol |
| alanine | arginine | choline | formate |
| alanine | arginine | choline | glycerol |
| alanine | arginine | choline | glycine |
| alanine | arginine | choline | leucine |
| alanine | arginine | choline | methanol |
| alanine | arginine | choline | methionine |
| alanine | arginine | choline | ornithine |
| alanine | arginine | choline | phenylalanine |
| alanine | arginine | choline | propylene |
| | | | glycol |
| alanine | arginine | choline | serine |
| alanine | arginine | choline | succinate |
| alanine | arginine | choline | threonine |
| arginine | choline | creatine | dimethylamine |
| arginine | choline | creatine | acetamide |
| arginine | choline | creatine | trimethylamine |
| arginine | choline | creatine | glutamine |
| arginine | choline | creatine | citrate |
| arginine | choline | creatine | ethanol |
| arginine | choline | creatine | formate |
| arginine | choline | creatine | glycerol |
| arginine | choline | creatine | glycine |
| arginine | choline | creatine | leucine |
| arginine | choline | creatine | methanol |
| arginine | choline | creatine | methionine |
| arginine | choline | creatine | ornithine |
| arginine | choline | creatine | phenylalanine |
| arginine | choline | creatine | propylene |
| | | | glycol |
| arginine | choline | creatine | serine |
| arginine | choline | creatine | succinate |
| arginine | choline | creatine | threonine |
| choline | creatine | dimethylamine | acetamide |
| choline | creatine | dimethylamine | trimethylamine |
| choline | creatine | dimethylamine | glutamine |
| choline | creatine | dimethylamine | citrate |
| choline | creatine | dimethylamine | ethanol |
| choline | creatine | dimethylamine | formate |
| choline | creatine | dimethylamine | glycerol |
| choline | creatine | dimethylamine | glycine |
| choline | creatine | dimethylamine | leucine |
| choline | creatine | dimethylamine | methanol |
| choline | creatine | dimethylamine | methionine |
| choline | creatine | dimethylamine | ornithine |
| choline | creatine | dimethylamine | phenylalanine |
| choline | creatine | dimethylamine | propylene |
| | | | glycol |
| choline | creatine | dimethylamine | serine |
| choline | creatine | dimethylamine | succinate |
| choline | creatine | dimethylamine | threonine |
| creatine | dimethylamine | acetamide | trimethylamine |
| creatine | dimethylamine | acetamide | glutamine |
| creatine | dimethylamine | acetamide | citrate |
| creatine | dimethylamine | acetamide | ethanol |
| creatine | dimethylamine | acetamide | formate |
| creatine | dimethylamine | acetamide | glycerol |
| creatine | dimethylamine | acetamide | glycine |
| creatine | dimethylamine | acetamide | leucine |
| creatine | dimethylamine | acetamide | methanol |
| creatine | dimethylamine | acetamide | methionine |
| creatine | dimethylamine | acetamide | ornithine |
| creatine | dimethylamine | acetamide | phenylalanine |
| creatine | dimethylamine | acetamide | propylene |
| | | | glycol |
| creatine | dimethylamine | acetamide | serine |
| creatine | dimethylamine | acetamide | succinate |
| creatine | dimethylamine | acetamide | threonine |
| dimethylamine | acetamide | trimethylamine | glutamine |
| dimethylamine | acetamide | trimethylamine | citrate |
| dimethylamine | acetamide | trimethylamine | ethanol |
| dimethylamine | acetamide | trimethylamine | formate |
| dimethylamine | acetamide | trimethylamine | glycerol |
| dimethylamine | acetamide | trimethylamine | glycine |
| dimethylamine | acetamide | trimethylamine | leucine |
| dimethylamine | acetamide | trimethylamine | methanol |
| dimethylamine | acetamide | trimethylamine | methionine |
| dimethylamine | acetamide | trimethylamine | ornithine |
| dimethylamine | acetamide | trimethylamine | phenylalanine |
| dimethylamine | acetamide | trimethylamine | propylene |
| | | | glycol |
| dimethylamine | acetamide | trimethylamine | serine |
| dimethylamine | acetamide | trimethylamine | succinate |
| dimethylamine | acetamide | trimethylamine | threonine |
| acetamide | trimethylamine | glutamine | citrate |
| acetamide | trimethylamine | glutamine | ethanol |
| acetamide | trimethylamine | glutamine | formate |
| acetamide | trimethylamine | glutamine | glycerol |
| acetamide | trimethylamine | glutamine | glycine |
| acetamide | trimethylamine | glutamine | leucine |
| acetamide | trimethylamine | glutamine | methanol |
| acetamide | trimethylamine | glutamine | methionine |
| acetamide | trimethylamine | glutamine | ornithine |
| acetamide | trimethylamine | glutamine | phenylalanine |
| acetamide | trimethylamine | glutamine | propylene |
| | | | glycol |
| acetamide | trimethylamine | glutamine | serine |
| acetamide | trimethylamine | glutamine | succinate |
| acetamide | trimethylamine | glutamine | threonine |
| trimethylamine | glutamine | citrate | ethanol |
| trimethylamine | glutamine | citrate | formate |
| trimethylamine | glutamine | citrate | glycerol |
| trimethylamine | glutamine | citrate | glycine |
| trimethylamine | glutamine | citrate | leucine |
| trimethylamine | glutamine | citrate | methanol |
| trimethylamine | glutamine | citrate | methionine |
| trimethylamine | glutamine | citrate | ornithine |
| trimethylamine | glutamine | citrate | phenylalanine |
| trimethylamine | glutamine | citrate | propylene |
| | | | glycol |
| trimethylamine | glutamine | citrate | serine |
| trimethylamine | glutamine | citrate | succinate |
| trimethylamine | glutamine | citrate | threonine |
| glutamine | citrate | ethanol | formate |
| glutamine | citrate | ethanol | glycerol |
| glutamine | citrate | ethanol | glycine |
| glutamine | citrate | ethanol | leucine |
| glutamine | citrate | ethanol | methanol |
| glutamine | citrate | ethanol | methionine |
| glutamine | citrate | ethanol | ornithine |
| glutamine | citrate | ethanol | phenylalanine |
| glutamine | citrate | ethanol | propylene |
| | | | glycol |
| glutamine | citrate | ethanol | serine |
| glutamine | citrate | ethanol | succinate |
| glutamine | citrate | ethanol | threonine |
| citrate | ethanol | formate | glycerol |
| citrate | ethanol | formate | glycine |
| citrate | ethanol | formate | leucine |
| citrate | ethanol | formate | methanol |
| citrate | ethanol | formate | methionine |
| citrate | ethanol | formate | ornithine |
| citrate | ethanol | formate | phenylalanine |
| citrate | ethanol | formate | propylene |
| | | | glycol |
| citrate | ethanol | formate | serine |
| citrate | ethanol | formate | succinate |
| citrate | ethanol | formate | threonine |
| ethanol | formate | glycerol | glycine |
| ethanol | formate | glycerol | leucine |
| ethanol | formate | glycerol | methanol |
| ethanol | formate | glycerol | methionine |
| ethanol | formate | glycerol | ornithine |
| ethanol | formate | glycerol | phenylalanine |
| ethanol | formate | glycerol | propylene |
| | | | glycol |
| ethanol | formate | glycerol | serine |
| ethanol | formate | glycerol | succinate |
| formate | glycerol | glycine | leucine |
| formate | glycerol | glycine | methanol |
| formate | glycerol | glycine | methionine |
| formate | glycerol | glycine | ornithine |
| formate | glycerol | glycine | phenylalanine |
| formate | glycerol | glycine | propylene |
| | | | glycol |
| formate | glycerol | glycine | serine |
| formate | glycerol | glycine | succinate |
| formate | glycerol | glycine | threonine |
| glycerol | glycine | leucine | methanol |
| glycerol | glycine | leucine | methionine |
| glycerol | glycine | leucine | ornithine |
| glycerol | glycine | leucine | phenylalanine |
| glycerol | glycine | leucine | propylene |
| | | | glycol |
| glycerol | glycine | leucine | serine |
| glycerol | glycine | leucine | succinate |
| glycerol | glycine | leucine | threonine |
| glycine | leucine | methanol | methionine |
| glycine | leucine | methanol | ornithine |
| glycine | leucine | methanol | phenylalanine |
| glycine | leucine | methanol | propylene |
| | | | glycol |
| glycine | leucine | methanol | serine |
| glycine | leucine | methanol | succinate |
| glycine | leucine | methanol | threonine |
| leucine | methanol | methionine | ornithine |
| leucine | methanol | methionine | phenylalanine |
| leucine | methanol | methionine | propylene |
| | | | glycol |
| leucine | methanol | methionine | serine |
| leucine | methanol | methionine | succinate |
| leucine | methanol | methionine | threonine |
| methanol | methionine | ornithine | phenylalanine |
| methanol | methionine | ornithine | propylene |
| | | | glycol |
| methanol | methionine | ornithine | serine |
| methanol | methionine | ornithine | succinate |
| methanol | methionine | ornithine | threonine |
| methionine | ornithine | phenylalanine | propylene |
| | | | glycol |
| methionine | ornithine | phenylalanine | serine |
| methionine | ornithine | phenylalanine | succinate |
| methionine | ornithine | phenylalanine | threonine |
| ornithine | phenylalanine | propylene | serine |
| | | glycol | |
| ornithine | phenylalanine | propylene | succinate |
| | | glycol | |
| ornithine | phenylalanine | propylene | threonine |
| | | glycol | |
| phenylalanine | propylene | serine | succinate |
| | glycol | | |
| phenylalanine | propylene | serine | threonine |
| | glycol | | |
| propylene | serine | succinate | threonine |
| glycol | | | |

Other representative combinations of 5, 6, 7, 8, 9, 10, 11, 12, 13, 14, 15, 16, 17, 18, 19, 20, 21, 22, 23, 24 or 25 metabolites can be readily determined by a skilled artisan through routine experimentation.

Similarly, metabolite combinations for determining a pregnant woman's risk of developing late-onset preeclampsia are determined in the same fashion as the ones described above for early-onset preeclampsia.

Thus, representative 2 metabolite combinations used to predict a pregnant woman's risk of developing late-onset preeclampsia include:

| 2 Metabolite Combinations | | 2 Metabolite Combinations | |
|---|---|---|---|
| 2-hydroxy- | acetamide | acetamide | acetate |
| butyrate | | | |
| 2-hydroxy- | acetate | acetamide | acetone |
| butyrate | | | |
| 2-hydroxy- | acetone | acetamide | carnitine |
| butyrate | | | |
| 2-hydroxy- | carnitine | acetamide | creatine |
| butyrate | | | |
| 2-hydroxy- | creatine | acetamide | creatinine |
| butyrate | | | |
| 2-hydroxy- | creatinine | acetamide | dimethylamine |
| butyrate | | | |
| 2-hydroxy- | dimethylamine | acetamide | glucose |
| butyrate | | | |
| 2-hydroxy- | glucose | acetamide | glycerol |
| butyrate | | | |
| 2-hydroxy- | glycerol | acetamide | propylene |
| butyrate | | | glycol |
| 2-hydroxy- | propylene | acetamide | ethylene glycol |
| butyrate | glycol | | |
| 2-hydroxy- | ethylene glycol | acetamide | threonine |
| butyrate | | | |
| 2-hydroxy- | threonine | acetamide | alanine |
| butyrate | | | |
| 2-hydroxy- | alanine | acetamide | trimethylamine |
| butyrate | | | |
| 2-hydroxy- | trimethylamine | acetamide | methylhistidine |
| butyrate | | | |
| 2-hydroxy- | methylhistidine | acetate | acetone |
| butyrate | | | |
| acetone | carnitine | acetate | carnitine |
| acetone | creatine | acetate | creatine |
| acetone | creatinine | acetate | creatinine |
| acetone | dimethylamine | acetate | dimethylamine |
| acetone | glucose | acetate | glucose |
| acetone | glycerol | acetate | glycerol |
| acetone | propylene | acetate | propylene |
| | glycol | | glycol |
| acetone | ethylene glycol | acetate | ethylene glycol |
| acetone | threonine | acetate | threonine |
| acetone | alanine | acetate | alanine |
| acetone | trimethylamine | acetate | trimethylamine |
| acetone | methylhistidine | acetate | methylhistidine |
| carnitine | creatine | creatine | creatinine |
| carnitine | creatinine | creatine | dimethylamine |
| carnitine | dimethylamine | creatine | glucose |
| carnitine | glucose | creatine | glycerol |
| carnitine | glycerol | creatine | propylene |
| | | | glycol |
| carnitine | propylene | creatine | ethylene glycol |
| | glycol | | |
| carnitine | ethylene glycol | creatine | threonine |
| carnitine | threonine | creatine | alanine |
| carnitine | alanine | creatine | trimethylamine |
| carnitine | trimethylamine | creatine | methylhistidine |
| carnitine | methylhistidine | creatinine | dimethylamine |
| dimethylamine | glucose | creatinine | glucose |
| dimethylamine | glycerol | creatinine | glycerol |
| dimethylamine | propylene | creatinine | propylene |
| | glycol | | glycol |
| dimethylamine | ethylene glycol | creatinine | ethylene glycol |
| dimethylamine | threonine | creatinine | threonine |
| dimethylamine | alanine | creatinine | alanine |
| dimethylamine | trimethylamine | creatinine | trimethylamine |
| dimethylamine | methylhistidine | creatinine | methylhistidine |
| glucose | glycerol | glycerol | propylene |
| | | | glycol |
| glucose | propylene | glycerol | ethylene glycol |
| | glycol | | |
| glucose | ethylene glycol | glycerol | threonine |
| glucose | threonine | glycerol | alanine |
| glucose | alanine | glycerol | trimethylamine |
| glucose | trimethylamine | glycerol | methylhistidine |
| glucose | methylhistidine | propylene | ethylene glycol |
| | | glycol | |
| ethylene glycol | threonine | propylene | threonine |
| | | glycol | |
| ethylene glycol | alanine | propylene | alanine |
| | | glycol | |
| ethylene glycol | trimethylamine | propylene | trimethylamine |
| | | glycol | |
| ethylene glycol | methylhistidine | propylene | methylhistidine |
| | | glycol | |
| threonine | alanine | alanine | trimethylamine |
| threonine | trimethylamine | alanine | methylhistidine |
| threonine | methylhistidine | trimethylamine | methylhistidine |

Representative 3 metabolite combinations used to predict a pregnant woman's risk of developing late-onset preeclampsia include:

| 3 Metabolite Combinations | | | 3 Metabolite Combinations | | |
|---|---|---|---|---|---|
| 2-hydroxy-butyrate | acetamide | acetate | acetamide | acetate | acetone |
| 2-hydroxy-butyrate | acetamide | acetone | acetamide | acetate | carnitine |
| 2-hydroxy-butyrate | acetamide | carnitine | acetamide | acetate | creatine |
| 2-hydroxy-butyrate | acetamide | creatine | acetamide | acetate | creatinine |
| 2-hydroxy-butyrate | acetamide | creatinine | acetamide | acetate | dimethylamine |
| 2-hydroxy-butyrate | acetamide | dimethylamine | acetamide | acetate | glucose |
| 2-hydroxy-butyrate | acetamide | glucose | acetamide | acetate | glycerol |
| 2-hydroxy-butyrate | acetamide | glycerol | acetamide | acetate | propylene glycol |
| 2-hydroxy-butyrate | acetamide | propylene glycol | acetamide | acetate | ethylene glycol |
| 2-hydroxy-butyrate | acetamide | ethylene glycol | acetamide | acetate | threonine |
| 2-hydroxy-butyrate | acetamide | threonine | acetamide | acetate | alanine |
| 2-hydroxy-butyrate | acetamide | alanine | acetamide | acetate | trimethylamine |
| 2-hydroxy-butyrate | acetamide | trimethylamine | acetamide | acetate | methylhistidine |
| 2-hydroxy-butyrate | acetamide | Methylhistidine | acetate | acetone | carnitine |
| acetone | creatine | creatinine | acetate | acetone | creatine |
| acetone | creatine | dimethylamine | acetate | acetone | creatinine |
| acetone | creatine | glucose | acetate | acetone | dimethylamine |
| acetone | creatine | glycerol | acetate | acetone | glucose |
| acetone | creatine | propylene glycol | acetate | acetone | glycerol |
| acetone | creatine | ethylene glycol | acetate | acetone | propylene glycol |
| acetone | creatine | threonine | acetate | acetone | ethylene glycol |
| acetone | creatine | alanine | acetate | acetone | threonine |
| acetone | creatine | trimethylamine | acetate | acetone | alanine |
| acetone | creatine | Methylhistidine | acetate | acetone | trimethylamine |
| acetone | creatine | creatinine | acetate | acetone | methylhistidine |
| creatine | creatinine | dimethylamine | creatinine | dimethylamine | glucose |
| creatine | creatinine | glucose | creatinine | dimethylamine | glycerol |
| creatine | creatinine | glycerol | creatinine | dimethylamine | propylene glycol |
| creatine | creatinine | propylene glycol | creatinine | dimethylamine | ethylene glycol |
| creatine | creatinine | ethylene glycol | creatinine | dimethylamine | threonine |
| creatine | creatinine | threonine | creatinine | dimethylamine | alanine |
| creatine | creatinine | alanine | creatinine | dimethylamine | trimethylamine |
| creatine | creatinine | trimethylamine | creatinine | dimethylamine | methylhistidine |
| creatine | creatinine | Methylhistidine | dimethylamine | glucose | glycerol |
| glucose | glycerol | propylene glycol | dimethylamine | glucose | propylene glycol |
| glucose | glycerol | ethylene glycol | dimethylamine | glucose | ethylene glycol |
| glucose | glycerol | threonine | dimethylamine | glucose | threonine |
| glucose | glycerol | alanine | dimethylamine | glucose | alanine |
| glucose | glycerol | trimethylamine | dimethylamine | glucose | trimethylamine |
| glucose | glycerol | Methylhistidine | dimethylamine | glucose | methylhistidine |
| glycerol | propylene glycol | ethylene glycol | propylene glycol | ethylene glycol | threonine |
| glycerol | propylene glycol | threonine | propylene glycol | ethylene glycol | alanine |
| glycerol | propylene glycol | alanine | propylene glycol | ethylene glycol | trimethylamine |
| glycerol | propylene glycol | trimethylamine | propylene glycol | ethylene glycol | methylhistidine |
| glycerol | propylene glycol | Methylhistidine | ethylene glycol | threonine | alanine |
| threonine | trimethylamine | Methylhistidine | ethylene glycol | threonine | trimethylamine |
| | | | ethylene glycol | threonine | methylhistidine |

Representative 4 metabolite combinations for predicting a pregnant woman's risk of developing late-onset preeclampsia include:

4 Metabolite Combinations

| | | | |
|---|---|---|---|
| 2-hydroxy- | acetamide | acetate | acetone |
| butyrate | | | |
| 2-hydroxy- | acetamide | acetate | carnitine |
| butyrate | | | |
| 2-hydroxy- | acetamide | acetate | creatine |
| butyrate | | | |
| 2-hydroxy- | acetamide | acetate | creatinine |
| butyrate | | | |
| 2-hydroxy- | acetamide | acetate | dimethylamine |
| butyrate | | | |
| 2-hydroxy- | acetamide | acetate | glucose |
| butyrate | | | |
| 2-hydroxy- | acetamide | acetate | glycerol |
| butyrate | | | |
| 2-hydroxy- | acetamide | acetate | propylene |
| butyrate | | | glycol |
| 2-hydroxy- | acetamide | acetate | ethylene glycol |
| butyrate | | | |
| 2-hydroxy- | acetamide | acetate | threonine |
| butyrate | | | |
| 2-hydroxy- | acetamide | acetate | alanine |
| butyrate | | | |
| 2-hydroxy- | acetamide | acetate | trimethylamine |
| butyrate | | | |
| 2-hydroxy- | acetamide | acetate | methylhistidine |
| butyrate | | | |
| acetamide | acetate | acetone | carnitine |
| acetamide | acetate | acetone | creatine |
| acetamide | acetate | acetone | creatinine |
| acetamide | acetate | acetone | dimethylamine |
| acetamide | acetate | acetone | glucose |
| acetamide | acetate | acetone | glycerol |
| acetamide | acetate | acetone | propylene |
| | | | glycol |
| acetamide | acetate | acetone | ethylene glycol |
| acetamide | acetate | acetone | threonine |
| acetamide | acetate | acetone | alanine |
| acetamide | acetate | acetone | trimethylamine |
| acetamide | acetate | acetone | methylhistidine |
| acetate | acetone | carnitine | creatine |
| acetate | acetone | carnitine | creatinine |
| acetate | acetone | carnitine | dimethylamine |
| acetate | acetone | carnitine | glucose |
| acetate | acetone | carnitine | glycerol |
| acetate | acetone | carnitine | propylene |
| | | | glycol |
| acetate | acetone | carnitine | ethylene glycol |
| acetate | acetone | carnitine | threonine |
| acetate | acetone | carnitine | alanine |
| acetate | acetone | carnitine | trimethylamine |
| acetate | acetone | carnitine | methylhistidine |
| acetone | carnitine | creatine | creatinine |
| acetone | carnitine | creatine | dimethylamine |
| acetone | carnitine | creatine | glucose |
| acetone | carnitine | creatine | glycerol |
| acetone | carnitine | creatine | propylene |
| | | | glycol |
| acetone | carnitine | creatine | ethylene glycol |
| acetone | carnitine | creatine | threonine |
| acetone | carnitine | creatine | alanine |
| acetone | carnitine | creatine | trimethylamine |
| acetone | carnitine | creatine | methylhistidine |
| carnitine | creatine | creatinine | dimethylamine |
| carnitine | creatine | creatinine | glucose |
| carnitine | creatine | creatinine | glycerol |
| carnitine | creatine | creatinine | propylene |
| | | | glycol |
| carnitine | creatine | creatinine | ethylene glycol |
| carnitine | creatine | creatinine | threonine |
| carnitine | creatine | creatinine | alanine |
| carnitine | creatine | creatinine | trimethylamine |
| carnitine | creatine | creatinine | methylhistidine |
| creatine | creatinine | dimethylamine | glucose |
| creatine | creatinine | dimethylamine | glycerol |
| creatine | creatinine | dimethylamine | propylene |
| | | | glycol |
| creatine | creatinine | dimethylamine | ethylene glycol |
| creatine | creatinine | dimethylamine | threonine |
| creatine | creatinine | dimethylamine | alanine |
| creatine | creatinine | dimethylamine | trimethylamine |
| creatine | creatinine | dimethylamine | methylhistidine |
| creatinine | dimethylamine | glucose | glycerol |
| creatinine | dimethylamine | glucose | propylene |
| | | | glycol |
| creatinine | dimethylamine | glucose | ethylene glycol |
| creatinine | dimethylamine | glucose | threonine |
| creatinine | dimethylamine | glucose | alanine |
| creatinine | dimethylamine | glucose | trimethylamine |
| creatinine | dimethylamine | glucose | methylhistidine |
| dimethylamine | glucose | glycerol | propylene |
| | | | glycol |
| dimethylamine | glucose | glycerol | ethylene glycol |
| dimethylamine | glucose | glycerol | threonine |
| dimethylamine | glucose | glycerol | alanine |
| dimethylamine | glucose | glycerol | trimethylamine |
| dimethylamine | glucose | glycerol | methylhistidine |
| dimethylamine | glucose | glycerol | propylene |
| | | | glycol |
| glucose | glycerol | propylene | ethylene glycol |
| | | glycol | |
| glucose | glycerol | propylene | threonine |
| | | glycol | |
| glucose | glycerol | propylene | alanine |
| | | glycol | |
| glucose | glycerol | propylene | trimethylamine |
| | | glycol | |
| glucose | glycerol | propylene | methylhistidine |
| | | glycol | |
| glucose | glycerol | propylene | propylene |
| | | glycol | glycol |
| glycerol | propylene | ethylene glycol | threonine |
| | glycol | | |
| glycerol | propylene | ethylene glycol | alanine |
| | glycol | | |
| glycerol | propylene | ethylene glycol | trimethylamine |
| | glycol | | |
| glycerol | propylene | ethylene glycol | methylhistidine |
| | glycol | | |
| glycerol | propylene | ethylene glycol | propylene |
| | glycol | | glycol |
| propylene | ethylene glycol | threonine | alanine |
| glycol | | | |
| propylene | ethylene glycol | threonine | trimethylamine |
| glycol | | | |
| propylene | ethylene glycol | threonine | methylhistidine |
| glycol | | | |
| propylene | ethylene glycol | threonine | propylene |
| glycol | | | glycol |
| ethylene glycol | threonine | alanine | trimethylamine |
| ethylene glycol | threonine | alanine | methylhistidine |
| ethylene glycol | threonine | alanine | propylene |
| | | | glycol |
| threonine | alanine | trimethylamine | methylhistidine |

Other 5, 6, 7, 8, 9, 10, 11, 12, 13, 14, 15, 16, 17 and 18 metabolite combinations for late-onset preeclampsia can be readily determined by a skilled artisan using routine experimentation.

Collection of blood from a woman is performed in accordance with the standard protocol hospitals or clinics generally follow. An appropriate amount of peripheral blood, e.g., between 3-20 ml, is collected (and stored, if needed) according to standard procedure prior to further preparation. In addition to whole blood, the serum of a woman's blood is suitable for use in the methods of the present invention and can be obtained by well known methods. For example, serum is obtained through centrifugation following blood clotting. Centrifugation is typically conducted at an appropriate speed, e.g., 1,500-3,000×g, in a chilled environment, e.g., at a temperature of about 4-10° C.

A dried blood sample from a pregnant woman can be used to measure metabolites for assessing the risk of a pregnant woman developing early-onset or late-onset preeclampsia. Blood is collected from a pregnant woman to be screened and transferred to filter paper where the blood dries, resulting in a spot, or spots, of dried blood on the filter paper. This method can also be used with other bodily fluids, including urine. For example, drops of urine, or other bodily fluid, from a pregnant woman can be placed on a specimen card and dried. The dried spots can then be analyzed by conventional immunological techniques or mass spectrometry, known to those of ordinary skill in the art, in a manner similar to the manner described herein with reference to the analysis of dried blood spots.

Analyzing dried blood samples or dried samples of other bodily fluids provides advantages in the transport and storage of such samples. By way of example, the dried blood spot samples on filter paper take up much less space than liquid blood samples in test tubes. Thus, less space is needed to store the samples, and the samples can be shipped by conventional mail or delivery services in small packages. A further advantage of using dried blood spots is that a smaller volume of blood is collected from the pregnant woman than in the case where a liquid blood sample is to be analyzed. Since less blood is needed, it is possible to make the collection technique less invasive, and potentially less painful for the pregnant woman. Other advantages in shipping dried blood or urine spots on filter paper in comparison with liquid blood samples in tubes or vials are readily apparent to those of skill in the art.

The filter paper for preparing dried samples, which is also referred to as a specimen collection card, is commercially available from a variety of sources, including Whatman, Inc., and Schleicher & Schuell. Generally a 3 inch by 4 inch, or a 5 inch by 7 inch card is utilized to collect the samples, however the filter paper may be any size that is convenient for transporting, storing and/or indexing the dried bodily fluid samples. The filter paper can be of sufficient size to enable a technician or nurse to write the pregnant woman's name or other identifier as well as other information such as the date the sample was collected on the paper. For example, the filter paper (specimen collection card) can be a Schleicher & Schuell #903® 3 inch by 4 inch card pre-printed with circles to provide locations for blood spots (application sites) and spaces to enter the patient's identification number, birth date, the date of collection of the sample and the physician's name. The filter paper can be provided with instructions and a lancet for a pregnant woman to collect her own blood.

The amount of blood taken from the pregnant woman should be sufficient to produce at least one spot on the filter paper approximately 10 millimeters in diameter. It is generally advantageous to produce more than one dried blood spot. Preferably, the amount of blood taken from the pregnant woman is sufficient to produce five to eight spots approximately 10 mm in diameter on the filter paper. It will be understood by those of ordinary skill in the art that the number of blood spots produced on a single piece of filter paper depends on the dimensions of the filter paper and the requirements of the physician and the clinical laboratory that will be analyzing the blood.

A variety of techniques for “spotting” blood on filter paper are known to the art. The choice of the particular technique utilized to produce the blood spots is a matter of choice to the person collecting the sample. Generally, a convenient site on the pregnant woman, preferably a finger tip, toe or ear lobe, is sterilized and then pricked with a sterile lancet. Lancets are commercially available from a variety of sources. An especially useful lancet is the Tenderlett® lancet manufactured and sold by Technidyne Corporation.

The drops of blood that form at the pricked site may be allowed to drip onto the filter paper to form the blood spots. Alternatively the pricked site may be placed in contact with the filter paper to produce the blood spots. The blood should dry on the filter paper prior to transport and/or storage.

The filter paper containing the blood spot is analyzed to determine the pregnant woman's level of one or more metabolites utilized in the screening protocol. The filter paper containing the blood spot may be stored and/or transported prior to analysis.

In addition to blood, other bodily fluids obtained from a pregnant woman being assessed for a risk of developing early-onset or late-onset preeclampsia can be used to measure concentrations of metabolites in such fluids. In one embodiment, the bodily fluid is urine, such as the first morning urine, which a pregnant woman can collect herself. The urine can be collected in cups specially provided for urine collection or can also be spotted on a filter paper, as discussed above.

In cases when urine is used as a bodily fluid, an easy-to-use nomogram can be used to translate a spot urine metabolite concentration into an estimated 24-hour excretion of one or more metabolites being used to test a pregnant woman for a risk of developing early-onset or late-onset preeclampsia.

The development of a set of easy-to-use nomograms provides a simple method for adjustment of a metabolite/creatinine ratio, which enables estimation of the 24-hour excretion of any metabolite with much greater accuracy than is possible without the adjustment. For example, the nomograms are used to adjust the ratio of the metabolite (e.g., glycerol) and creatinine concentrations to the estimated 24-hour creatinine excretion based upon one or more characteristics of the individual, such as age, gender, race, weight, lean body mass, muscle mass, adiposity, physical activity, or any combination thereof, to better estimate the 24-hour metabolite excretion.

A spot urine sample is obtained from a pregnant woman being assessed for a risk of developing early-onset or late-onset preeclampsia by any standard methods known in the art. The concentrations of a metabolite of interest and creatinine in the spot urine sample are determined by any of the methods described herein, such as mass spectroscopy or NMR. In cases of well established metabolites, the measurement of the metabolite concentration can be obtained from a standard clinical laboratory or a dipstick, if such is available. Then, a standard value for estimated 24-hour urine creatinine excretion is selected from an array of such standard values for 24-hour urine creatinine excretion. The values in the array are obtained from a nomogram that is a product of an equation that estimates 24-hour creatinine excretion from variables including subject's age, gender, race, weight, muscle mass, lean body mass, muscle mass, adiposity, physical activity, or a combination thereof. A standard value for estimated 24-hour excretion of the metabolite is then selected from an array of such standard values for 24-hour metabolite excretion. The values in the array are based upon the standard value for estimated 24-hour urine creatinine excretion determined in the previous step, the metabolite concentration and the creatinine concentration.

The mass of creatinine excreted by a pregnant woman being tested, who is not afflicted with any substantial challenge to homeostasis can be expected to remain reasonably constant over time. In other words, for any individual with stable renal function, the 24-hour urinary creatinine excretion is constant from day to day. This applies as well to individuals with impaired renal function, provided it is stable.

Determination of the amount of a metabolite that is excreted in the urine, or of changes in the amount excreted, is generally obtained by measuring the concentration of the metabolite in a 24-hour urine collection. For many metabolites, more convenient estimation of their excretion can be obtained by estimation of their excretion from a spot urine sample by assessing their concentration in the urine relative to the concentration of creatinine in that sample.

The 24-hour creatinine excretion, although constant from day to day in any given individual, differs considerably between individuals. Thus, for example, a 100 pound woman might have a urine creatinine excretion of 900 mg/day, whereas a 250 pound male might have a urine creatinine excretion of 2500 mg/day. Because of this considerable between-person variance in 24 hour urine creatinine excretion, to accurately estimate the 24-hour excretion of a metabolite from a metabolite/creatinine ratio, the ratio must be adjusted to take into account the amount of creatinine typically excreted by that individual in 24-hours. The usual method of determining 24-hour creatinine excretion, again, is a 24-hour urine collection, which is plagued by the impracticality of collecting urine for 24 hours and by inaccuracy in many cases due to under-collection. However, an estimate of the individual's 24-hour creatinine excretion, without any urine collection, is possible if one or more variables associated with between-subject variation in creatinine excretion are accounted for in determining the estimate of creatinine excretion. Such variables include lean muscle mass, which can be largely determined from gender, race, age, weight, muscularity or a combination thereof. The estimated 24-hour creatinine thus takes into account between-person differences, requires no urine collection, and, unlike actual 24-hour urine collections, its accuracy does not suffer from incomplete collections. The estimated 24-hour creatinine excretion determined in this fashion is used to adjust the measured metabolite/creatinine ratio to accurately predict 24-hour excretion of that metabolite.

This method provides an estimate for 24-hour urine creatinine and simple instructions for use to enable appropriate adjustment of the metabolite/creatinine ratio. It avoids the need for any blood sample, or for measurement of urine volume, while adding precision to raw metabolite/creatinine ratios. The adjusted estimate of 24-hour creatinine excretion is available in the form of databases, look-up tables or nomograms, for example. In preferred embodiments, these values are arrayed such that a “standard” value for a given subject, depending upon the subject's age, race, gender, weight, muscle mass, lean body mass and/or level of physical activity, can be selected from the array. Without wishing to be bound by a particular theory, this method of estimating 24-hour urine creatinine is thought to improve the accuracy of prediction of metabolite excretion because it bears a strong relationship to an individual's muscle mass, which is the source of creatinine. Since weight, gender and ethnicity are prominent determinants of total muscle mass, and are readily available measures, formulae can be created for estimating a subject's 24-hour creatinine excretion. An exemplary formula, is: {y=1150 mg−407.4 mg (if female)+(5.7)(weight in pounds)−88 mg (if white)} wherein y is a subject's estimated 24-hr creatinine excretion in mg. It will be appreciated that the artisan can readily refine the model by adding variables (e.g., gender, age, ethnicity such as Asian, Caucasian or African, lean muscle mass, adiposity, or level of physical activity), accumulating data on each variable, and deriving therefrom, by well-known methods of regression analysis, more powerful regression formulae.

Any equation so determined can be applied to estimate the 24-hour creatinine excretion of any individual, without limitation, by hand, or by means of a computer program, a look-up table or a nomogram. A nomogram is preferred since it can be used without the need for calculations by the subject.

The adjusted estimate of a pregnant woman's 24-hour excretion of one or more metabolites is thus readily calculated in two steps: first, by consulting the table of estimated 24-hour creatinine excretion values for pregnant women, and selecting a value corresponding to the individual's weight and age, and second, by using the selected value along with the values found for urinary metabolite concentration and urinary creatinine concentration in a spot urine sample, solving the following equation: Subject's Metabolite Excretion=((Creatinine Excretion per Table) (Metabolite Conc.))/((Creatinine Conc.)×10). Again, the equation can be solved, without limitation, by hand, or by means of a computer program, a look-up table or a nomogram.

The concentrations (or levels) of metabolites obtained from a bodily fluid of a pregnant woman being assessed for a risk of developing early-onset or late-onset preeclampsia can be measured using a variety of techniques well known in the art. Such methods include, but are not limited to, mass spectrometry (MS), nuclear magnetic resonance (NMR), immunoblot analysis, immunohistochemical methods (e.g., in situ methods based on antibody detection of metabolites), and immunoassays (e.g., ELISA). It should be noted that a method used to measure metabolites in a pregnant woman being assessed for a risk of developing early-onset or late-onset preeclampsia is the same method that is used to measure concentrations of corresponding metabolites in control subjects (i.e., pregnant women with early-onset or late-onset preeclampsia, depending on the condition being tested, and pregnant women exhibiting normal blood pressure and normal protein levels in urine).

Mass spectrometry (e.g., electrospray ionization or ESI mass spectrometry) can be used, for example, to determine the concentrations of metabolites in a maternal sample. In general, mass spectrometry involves ionizing a sample containing one or more molecules of interest, and then m/z separating and detecting the resultant ions (or product ions derived therefrom) in a mass analyzer, such as, without limitation, a quadrupole mass filter, quadrupole ion trap, time-of-flight analyzer, FT/ICR analyzer or Orbitrap, to generate a mass spectrum representing the abundances of detected ions at different values of m/z. See, e.g., U.S. Pat. Nos. 6,204,500, 6,107,623, 6,268,144, and 6,124,137, and articles, such as Wright et al., Prostate Cancer and Prostatic Diseases 2:264-76 (1999); and Merchant and Weinberger, Electrophoresis 21:1164-67 (2000), each of which is hereby incorporated by reference in its entirety.

Tandem mass spectrometry (e.g., using a quadrapole mass spectrometer) can be employed in the methods of the invention. As used herein “tandem mass spectrometry,” or “MS/MS” refers to a technique wherein a precursor ion or group of ions generated from a molecule (or molecules) of interest may be isolated or selected in an MS instrument, and these precursor ions subsequently fragmented to yield one or more fragment ions that are then analyzed in a second MS procedure. By careful selection of precursor ions, ions produced by certain metabolites of interest are selectively passed to the fragmentation chamber, where collision with atoms or molecules of an inert gas occurs to produce the fragment ions. Since both the precursor and fragment ions are produced in a reproducible fashion under a given set of ionization/fragmentation conditions, the MS/MS technique can provide an extremely powerful analytical tool. For example, the combination of filtration/fragmentation can be used to eliminate interfering substances, and can be particularly useful in complex samples, such as biological samples.

Ions can be produced using a variety of methods including, but not limited to, electrospray ionization (“ESI”), and matrix-assisted laser desorption ionization (“MALDI”).

Electrospray ionization, or ESI, mass spectrometry can be used to determine the expression level of one or more metabolites in a maternal sample. The term “electrospray ionization,” or “ESI,” as used herein refers to methods in which a solution is passed along a short length of capillary tube, to the end of which is applied a high positive or negative electric potential. Solution reaching the end of the tube is vaporized (nebulized) into a jet or spray of very small droplets of solution in solvent vapor. This mist of droplets flows through an evaporation chamber which may be heated to prevent condensation and to evaporate solvent. As the droplets get smaller, the electrical surface charge density increases until such time that the natural repulsion between like charges causes ions as well as neutral molecules to be released.

For MALDI, the sample is mixed with an energy-absorbing matrix, which facilitates desorption of analyte (metabolite) molecules.

In methods such as MS/MS, where precursor ions are isolated for further fragmentation, collision-induced dissociation (“CID”) is often used to generate the fragment ions for further detection. In CID, precursor ions undergo fragmentation induced by energetic collisions with neutral molecules or atoms. Sufficient energy must be deposited in the precursor ion so that certain bonds within the ion can be broken due to increased vibrational energy.

NMR spectroscopy can also be used to determine concentrations of metabolites in a pregnant woman's sample who is being tested for early-onset or late-onset preeclampsia, and in samples which are used as controls. NMR is based on the magnetic properties of the nucleus of the constituent atoms that make up the metabolite. Exposure to radiofrequency (RF) energy will result in a change of energy state or orientation of these ‘nuclear magnets’. The exact frequency of RF energy needed to achieve this change in energy state is specific for a particular atomic element (Bothwell J H, Griffin J L. Biol Rev Camb Philos Soc. 2010 Oct. 24. doi: 10.1111/j.1469-185X.2010.00157.x.). When the RF energy pulse is turned off the nuclei returns to their resting position, thereby remitting the stored energy in the form of RF waves. The parameters of the RF waves emitted from the nuclei provides information on the chemical substances that are present in the sample being tested. The emitted RF waves are read as a plot of intensity on the Y-axis and frequency on the X-axis. These spectra are compared to internal standard substances placed in the specimen and existing databases to determine the identity and concentrations of metabolites within the specimens being tested. In some embodiments, the metabolite concentrations are analyzed using ¹H NMR.

The biological samples can be subjected to one or more sample preparation steps prior to analysis by mass spectrometry. For example, a serum sample can be enriched for target metabolites of interest using techniques known in the art, such as by concentrating the samples.

In some embodiments, samples are subjected to a liquid chromatography (LC) purification step prior to mass spectrometry. Methods of coupling liquid chromatography techniques to MS analysis are well known and widely practiced in the art. Traditional LC analysis relies on the chemical interactions between sample components and column packings, where laminar flow of the sample through the column is the basis for separation of the analyte of interest from the test sample. The skilled artisan will understand that separation in such columns is a diffusional process. Numerous column packings are available for chromatographic separation of samples, and selection of an appropriate separation protocol is an empirical process that depends on the sample characteristics, the metabolite of interest, the interfering substances present and their characteristics, etc. Various packing chemistries can be used depending on the needs (e.g., structure, polarity, and solubility of compounds being purified). For example, the columns can be polar, ion exchange (both cation and anion), hydrophobic interaction, phenyl, C-2, C-8, C-18 columns, polar coating on porous polymer, or others that are commercially available. During chromatography, the separation of materials is effected by variables such as choice of eluant (also known as a “mobile phase”), choice of gradient elution and the gradient conditions, temperature, etc.

A metabolite may be purified by applying a sample to a column under conditions where the metabolite of interest is reversibly retained by the column packing material, while one or more other materials are not retained. A first mobile phase condition can be employed where the metabolite of interest is retained by the column, and a second mobile phase condition can subsequently be employed to remove retained material from the column, once the non-retained materials are washed through. Alternatively, a metabolite can be purified by applying a sample to a column under mobile phase conditions where the metabolite of interest elutes at a differential rate in comparison to one or more other materials. As discussed above, such procedures can enrich the amount of one or more metabolites of interest relative to one or more other components of the sample.

Once the mass spectrometric or NMR analysis of the prepared sample has been completed, the quantities of the metabolites in the sample can be determined by integration of the relevant mass spectral peak areas, as known in the prior art. When isotopically-labeled internal standards are used, as described above, the quantities of the metabolites of interest are established via an empirically-derived or predicted relationship between metabolite quantity (which may be expressed as concentration) and the area ratio of the metabolite and internal standard peaks at specified transitions. Other implementations of the assay can utilize external standards or other expedients for metabolite quantification.

It is obvious to those skilled in the art that a cut-off can be established to determine whether a patient is at increased risk of developing early-onset or late-onset preeclampsia. This cut-off may be established by the laboratory, the physician or on a case by case basis by each patient. The cut-off level can be based on several criteria including the average risk of developing early-onset or late-onset preeclampsia, race, maternal weight or other criteria known to those skilled in the art. The cut-off level could be established using a number of methods, including: percentiles, mean plus or minus standard deviation(s); multiples of median value; patient specific risk or other methods known to those skilled in the art.

For the purposes of the discriminant analysis, an assumption is made as to the prior probability of developing early-onset or late-onset preeclampsia. For the multivariate discriminant analysis a decision is made as to what risk cutoff level constitutes a positive test result.

If a positive test result is indicated, the patient may be counseled about potential prophylactic therapy, such as the use of aspirin to prevent preeclampsia or reduce its negative effects on both the fetus and mother.

As obvious to one skilled in the art, in any of the embodiments discussed above, changing the risk cut-off level of a positive test or using different a priori risks which may apply to different subgroups in the population could change the results of the discriminant analysis for each patient. Accordingly, if by the methods of the present invention a pregnant woman being assessed for a risk of developing early-onset or late-onset preeclampsia has a greater probability than the cut-off value, she may be advised to undergo prophylactic therapy to either prevent or reduce the effects of preeclampsia.

The methods for predicting a pregnant woman's risk of developing early-onset or late-onset preeclampsia can include analysis of factors other than metabolite concentrations in determining such risk. These factors include, without limitation, the crown-to-rump length of the fetus (CRL), which is a precise measure of the age of the fetus, and/or Doppler's ultrasound measurement of the pregnant woman's uterine artery blood flow resistance.

Accordingly, the present invention is also directed to a method for determining a pregnant woman's risk for developing early-onset preeclampsia, wherein the method comprises measuring concentrations of one or more metabolites selected from the group consisting of acetate, alanine, arginine, choline, creatine, dimethylamine, acetamide, trimethylamine, glutamine, citrate, ethanol, formate, glycerol, glycine, leucine, methanol, methionine, ornithine, phenylalanine, propylene glycol, serine, succinate, hydroxy-isovalerate, pyruvate, hydroxy-buturate and threonine, in the pregnant woman's bodily fluid. The pregnant woman's one or more metabolite concentrations are compared to the corresponding one or more metabolite concentrations obtained from pregnant women with early-onset preeclampsia and to the corresponding one or more metabolite concentrations obtained from pregnant women exhibiting normal blood pressure and normal protein levels in urine, which have been standardized according to the control fetal crown rump length average values. Crown rump length (CRL) of the pregnant woman's fetus is measured, and her metabolite concentrations are then compared to standardized control metabolite concentrations based on CRL values. The pregnant woman's risk of developing early-onset preeclampsia is predicted, wherein a statistically significant change in concentration of one or more metabolites between the pregnant woman and the corresponding one or more standardized metabolites from the pregnant women exhibiting normal blood pressure and normal protein levels in urine indicates a greater probability of developing early-onset preeclampsia.

The crown-rump length (CRL) is an ultrasound measurement of the length of the first trimester fetus. It is the distance from the top of the head to the buttock in the midline. The measurement provides a very accurate determination of the gestational age of the first trimester fetus, and has been shown to be accurate to within 3-5 days of the gestational age in women in which the exact time of embryonic implantation is known, namely women undergoing in vitro fertilization (IVF) pregnancies. Measurement of the CRL in the first trimester is more accurate than widely used methods of gestational age assignment, such as the date of the last menstrual period (LMP). Measuring CRL of the pregnant woman's fetus is performed at a gestational age from 8 weeks to 13⁺ weeks, and preferably from 11⁺⁰ to 13⁺⁶ weeks of gestation. The correlation between CRL values and gestational age is well established in the art, and is readily available.

For example, CRL can be used to standardize the measurement of creatine and choline concentrations or acetate, glycerol and hydroxyisovalerate concentrations in the pregnant woman's bodily fluid for predicting a risk of a pregnant woman developing early-onset preeclampsia. LMP based gestational age may be also used for standardization; however, this is generally considered to be less reliable than CRL based standardization.

CRL can also be used to standardize the measurement of creatine concentration in the pregnant woman's bodily fluid for predicting a risk of a pregnant woman developing early-onset preeclampsia.

CRL can also be combined with metabolite concentrations for purposes of predicting a risk of a pregnant woman developing late-onset preeclampsia. A method for determining a pregnant woman's risk for developing late-onset preeclampsia comprises measuring concentrations of one or more metabolites selected from the group consisting of 2-hydroxy-butyrate, acetamide, acetate, acetone, carnitine, creatine, creatinine, dimethylamine, glucose, glycerol, propylene glycol, ethylene glycol, threonine, alanine, trimethylamine, 3-hydroxy-butyrate, valine, pyruvate and methylhistidine in the pregnant woman's bodily fluid. The pregnant woman's one or more metabolite concentrations are compared to the corresponding one or more metabolite concentrations obtained from pregnant women with late-onset preeclampsia and to the corresponding one or more metabolite concentrations obtained from pregnant women exhibiting normal blood pressure and normal protein levels in urine, which have been standardized according to the control fetal CRL average values. CRL of the pregnant woman's fetus is measured, and her metabolite concentrations are then compared to standardized control metabolite concentrations based on CRL values. The pregnant woman's risk of developing late-onset preeclampsia is predicted, wherein a statistically significant change in concentration of one or more metabolites between the pregnant woman and the corresponding one or more standardized metabolites from the pregnant women exhibiting normal blood pressure and normal protein levels in urine indicates the probability of developing late-onset preeclampsia.

Doppler ultrasound measurements of a pregnant woman's uterine artery blood flow resistance represented by uterine artery Doppler pulsatility index (UtPI (MOM)) can be combined with metabolite concentrations for predicting a pregnant woman's risk of developing early-onset preeclampsia.

A method for determining a pregnant woman's risk for developing early-onset preeclampsia comprises measuring concentrations of one or more metabolites selected from the group consisting of acetate, alanine, arginine, choline, creatine, dimethylamine, acetamide, trimethylamine, glutamine, citrate, ethanol, formate, glycerol, glycine, leucine, methanol, methionine, ornithine, phenylalanine, propylene glycol, serine, succinate, hydroxy-isovalerate, pyruvate, hydroxy-butyrate and threonine in the pregnant woman's bodily fluid. The pregnant woman's one or more metabolite concentrations are compared to the corresponding one or more metabolite concentrations obtained from pregnant women with early-onset preeclampsia and to the corresponding one or more metabolite concentrations obtained from pregnant women exhibiting normal blood pressure and normal protein levels in urine. All metabolite concentrations are measured at the same or similar gestational age. The pregnant woman's uterine artery blood flow resistance is measured. The pregnant woman's uterine artery blood flow resistance is compared to the uterine artery blood flow resistances of pregnant women with early-onset preeclampsia and to the uterine artery blood flow resistances of pregnant women exhibiting normal blood pressure and normal protein levels in urine. The pregnant woman's risk of developing early-onset preeclampsia is predicted, wherein a statistically significant change in concentration of one or more metabolites between the pregnant woman and the corresponding one or more metabolites from the pregnant women exhibiting normal blood pressure and normal protein levels in urine and a statistically significant uterine artery blood flow resistance difference between the pregnant woman and the pregnant women exhibiting normal blood pressure and normal protein levels in urine indicate a greater probability of developing early-onset preeclampsia.

Doppler sound measurements of uterine artery blood flow resistance can be taken during the first trimester, such as at a gestational age from 8 weeks to 14 weeks, and preferably from 10 to 13 weeks.

It should be noted that in cases when metabolite measurements are combined with either CRL information or UtPI (MOM), the particular order in which metabolite measurements and CRL or UtPI (MOM) are taken is unimportant, as long as the measurements are taken in the gestational weeks specified herein.

Metabolites used to predict either early-onset or late-onset preeclampsia can also be standardized according to a number of different variables such as maternal race, maternal weight, presence of maternal medical disorders such as diabetes mellitus or hypertension, age, smoking, and the like.

The particulars for performing the analyses involved in evaluating a pregnant woman's risk of developing early-onset or late-onset preeclampsia are described in the Examples. By way of example and not of limitation, the calculation of probability of a pregnant woman developing early-onset or late-onset preeclampsia can be derived using logistic regression analysis. In this mathematical equation, a number of potential predictor variables which can either be numerical (age in years, body weight, body mass index (BMI)) or categorical (e.g. race/ethnicity, the presence of other disorders such as diabetes or chronic hypertension) are analyzed to find the optimal combination of variables that will most accurately predict an outcome of interest, e.g. chromosomal abnormality. The results of the logistic regression analysis can be converted to a format that expresses the probability of the particular outcome. Such formulas for predicting a risk of developing early-onset or late-onset preeclampsia are shown in the Examples.

Control samples are appropriately matched to the pregnant women being assessed for a risk of developing early-onset or late-onset preeclampsia as is standard in the art. As such, the bodily fluids obtained from pregnant women being tested and controls (pregnant women with early-onset preeclampsia or late-onset preeclampsia, depending on the condition being tested, and pregnant women exhibiting normal blood pressure and normal protein levels in urine) are preferably the same (e.g., blood vs. blood), the gestational age of pregnant women being tested and controls is the same or similar (i.e., in the same range (e.g., 10-13 weeks of gestation)), concentrations of metabolites are measured using the same techniques, etc.

“Normalized” or “standardized” refers to data mathematically adjusted by a factor such that the elements of the factored dataset are more readily compared than the elements of the unfactored dataset. For example, each normalized metabolite concentration value can be an estimated average (mean or median) of observed metabolite concentrations from a population of pregnant women exhibiting normal blood pressure and normal protein levels in urine and having similar CRL values or being of similar age, race, weight, BMI, etc. Additionally, metabolite concentrations can be standarized by introducing a particular variable, such as CRL, maternal weight, presence of medical disorders, age, smoking, etc. into an appropriate regression analysis equation as shown in the Examples. These methods of standardization are well known to one of ordinary skill in the art.

Another aspect of the invention is an article of manufacture such as a computer readable medium encoded with machine-readable data and/or a set of instructions, where the instructions can be carried out by a computer or a processing system. Such a computer readable medium can be a conventional compact disk read only memory (CD-ROM) or a rewritable medium such as a magneto-optical disk which is optically readable and magneto-optically writable. The computer readable medium can be prepared by available procedures. For example, the computer readable medium can have a suitable conventional substrate and a suitable conventional coating, usually on one side of the substrate.

In the case of CD-ROM, as is well known, a reflective coating can be employed that is impressed with a plurality of pits to encode the machine-readable data. The arrangement of pits is read by reflecting laser light off the surface of coating. A protective coating, which preferably is substantially transparent, is used on top of coating that has a plurality of pits.

In the case of a magneto-optical disk, as is well known, the coating has no pits, but has a plurality of magnetic domains whose polarity or orientation can be changed magnetically when heated above a certain temperature, as by a laser. The orientation of the domains can be read by measuring the polarization of laser light reflected from coating. The arrangement of the domains encodes data, for example, normalized metabolite concentration values measured in a bodily fluid of pregnant women, such as blood or urine, as described above.

Data capable of facilitating determination of the risk that a pregnant woman will develop early-onset or late onset preeclampsia is stored in a machine-readable storage medium. Executable code can also be included in the machine-readable medium that is capable of predicting the pregnant woman's risk of developing preeclampsia when the medium is used in conjunction with a computer or processor. For example, the machine readable medium, used in conjunction with a computer or processor can determine a pregnant woman's likelihood of developing preeclampsia after an individual enters data relating to the pregnant woman's bodily fluid concentrations of one or more metabolites.

The term “article of manufacture” as used herein refers to a kit or a computer readable medium (e.g., computer chip or magnetic storage medium such as hard disk drives, floppy disks, tape), optical storage medium (e.g., OD-ROMs, optical disks, etc.), volatile and non-volatile memory devices (e.g., EEPROMs, ROMs, PROMs, RAMs, DRAMs, SPAMs, firmware. programmable logic, etc.). Code and data in the computer readable medium is accessed and executed by a processor. The code and/or data in which implementations are made may further be accessible through a transmission media or from a file server over a network. Of course, those skilled in the art will recognize that many modifications may be made to this configuration without departing from the scope of the implementations and that the article of manufacture may comprise any information bearing medium known in the art.

The article of manufacture and the computer-readable medium are non-transitory, such that they comprise all such articles of manufacture and computer-readable media except for a transitory, propagating signal.

Having described the invention in detail, it will be apparent that modifications and variations are possible without departing from the scope of the invention defined in the appended claims.

### EXAMPLES

The following non-limiting examples are provided to further illustrate the present invention.

### NMR Sample Preparation

Plasma samples contain a substantial portion of large molecular weight proteins and lipoproteins, which can affect the identification and quantification of small molecule metabolites by NMR spectroscopy. To prevent this, a step was introduced into the protocol to remove plasma proteins (deproteinization). There are several routes to plasma deproteinization, including organic solvent (acetonitrile, methanol, isopropanol) precipitation, as well as diffusion editing. An ultrafiltration protocol similar to that described by Tiziani et al. (Tiziani S et al., Anal Biochem. 2008; 377:16-23; Weljie A M et al., Anal Chem. 2006; 78:4430-42) yielded excellent spectra resulting in metabolite concentrations that closely matched known values measured using standard clinical chemistry techniques. Ultrafiltration also has other advantages: it is relatively quick, very reproducible, does not introduce unwanted solvent peaks and is “safe” in terms of avoiding unwanted side-reactions with biofluid metabolites.

Prior to filtration, 3 KDa cut-off centrifugal filter units (Amicon Microcon YM-3) were rinsed three times each with 0.5 mL of water and centrifuged at 10,000 rpm for 30 minutes to remove residual glycerol bound to the filter membranes. 350 μL aliquots of each plasma sample were then transferred into the centrifuge filter devices. The samples were then spun at a rate of 10,000 rpm for 20 minutes to remove macromolecules (primarily proteins and lipoproteins) from the sample. The subsequent filtrates were then checked visually as an indication that the membrane was compromised. For those samples where the membrane was compromised, the filtration process was repeated with a different filter, and the filtrate was inspected again. The subsequent filtrates were collected and the volumes were recorded. If the total volume of the sample was under 300 μL, an appropriate amount from a 50 mM monobasic sodium phosphate buffer (pH 7) was added to the sample until the total volume was 300 μL. Subsequently, 35 μL of deuterium oxide and 15 μL of a standard buffer solution (11.667 mM DSS [disodium-2,2-dimethyl-2-silapentane-5-sulphonate], 730 mM imidazole, and 0.47% NaN₃ in water) were added to the sample. The plasma samples (350 μL) were then transferred to a standard Shigemi microcell NMR tube for subsequent spectral analysis.

### NMR Spectroscopy

All ¹H-NMR spectra were collected on a 500 MHz Inova (Varian Inc., Palo Alto, Calif.) spectrometer equipped with either a 5 mm HCN Z-gradient or PFG Varian cold-probe. ¹H-NMR spectra were acquired at 25° C. using the first transient of the NOESY-presaturation pulse sequence, which was chosen for its high degree of quantitative accuracy. Spectra were collected with 256 transients and 8 steady state scans using a 4 second acquisition time and a 1.5 second recycle delay.

### NMR Compound Identification and Quantification

All free induction decays (FIDs) were zero-filled to 64 k data points and subjected to line broadening of 0.5 Hz. The singlet produced by the DSS methyl groups was used as an internal standard for chemical shift referencing (set to 0 ppm) and for quantification. All ¹H-NMR spectra were processed and analyzed using the Chenomx NMR Suite Professional software package version 7.0 (Chenomx Inc., Edmonton, AB). The Chenomx NMR Suite software allows for qualitative and quantitative analysis of an NMR spectrum by manually fitting spectral signatures from an internal database to the spectrum. Specifically, the spectral fitting for each metabolite was done using the standard Chenomx 500 MHz metabolite library. Typically, 90% of all visible peaks were assigned to a compound, and more than 90% of the spectral area could be routinely fit using the Chenomx spectral analysis software. Most of the visible peaks were annotated with a compound name. It has been previously shown that this fitting procedure provides absolute concentration accuracies of 90% or better. Each spectrum was processed and analyzed by at least two NMR spectroscopists to minimize compound misidentification and misquantification. Sample spiking was used to confirm the identities of assigned compound. Sample spiking involved the addition of 20-200 μM of the suspected compound to selected clostridium samples and examination of whether the relative NMR signal intensity changed as expected.

### Statistical Analysis of Metabolic Data and Data Normalization

The data were adjusted to achieve a normal or Gaussian distribution of metabolite concentrations. This permits the use of certain statistical tests that require normal distribution. A technique called Pareto scaling was used to achieve normalization. Metabolite concentrations were mean-centered, i.e. expressed with reference to the overall mean value and then divided by the standard deviation of each of the metabolites.

Principle components analysis (PCA) was used as a statistical technique for reducing (“dimensional reduction”) the number of metabolites (from a large original group of metabolites) that significantly account for the variance (difference) between the diseased and normal groups to a small number.

Principle component analysis (PCA) is an unsupervised classification technique for transforming a complex collection of data points such that the important properties of the sample can be more simply displayed along the X- and Y-axes. PCA involves calculating correlation coefficients between sets of data and then determining eigenvalues and eigenvectors through linear algebraic transformations. The result is a set of “vectors” of different metabolites which can be used to plot out the metabolite data on an X-Y cluster plot. The first, and most significant vector is called the 1^{st} principal component (X axis) and the second most significant vector is called the 2^{nd} principal component (Y axis). If the data are separable it should be possible to see two sets of clusters. Two clusters indicate that there are some significant metabolic or metabolite differences between the two sets of samples i.e. disease versus normals. The predictors are displayed on a 2- or 3-dimensional rather than on a high dimensional (>3) chart that would be required for the large number of metabolites available. Each principal component (metabolite set) displayed accounts for a significant percentage of the variance between the groups being studied e.g. early-onset preeclampsia vs. normal group or late-onset preeclampsia vs. normal group. Often a small number of principal components can account for a high percentage (e.g. >90%) of the variance between the two groups. In such circumstances, plotting the information on a three dimensional graph represents an easy means of visualizing the separation between the groups (Sumner L W et al., Methods Mol Biol. 2007; 406:409-36).

A Predictive Model for Preeclampsia: Mean (SD) metabolite concentrations in early-onset preeclampsia vs. controls were compared. Stepwise logistic regression analyses were performed with early-onset or late-onset preeclampsia as the dependent variable and metabolites as the independent or determinant variables. Other variables including fetal CRL and maternal demographic and medical status (racial origin, weight, height, smoking, method of conception, previous pregnancy with or without preeclampsia (PE), diabetes mellitus, chronic hypertension) were combined with metabolite concentrations and run in this regression analyses. Finally, regression analyses including first trimester uterine artery PI and the preceding metabolomic and other maternal markers were performed for the prediction of early-PE. Based on these analyses, several regression equations for predicting the individual risk of early-PE were developed. Individual risk or probability of early-PE was calculated for each patient in the study. Different probability thresholds (e.g. individual probability of early-PE> 1/10, > 1/20, > 1/30 etc.) were each used serially to define an increased risk of early-PE. Using each cut-off value as a screening test, paired sensitivity (defined as the percentage of early-PE cases with probability value above this threshold) and specificity (percentage of normal cases with calculated probability of having early-PE below this threshold), were calculated. False positive rate (FPR), defined as 1-specificity, could then be easily determined from the specificity value. Using multiple different probability threshold values, a series of paired sensitivity and FPR values were generated. Thereafter, a ROC curve was plotted with sensitivity values on the Y-axis and the corresponding FPR on the X-axis. The area (AUC) under the receiver-operator characteristic (ROC) curve indicates the accuracy of a test for correctly identifying a disorder i.e. early-PE cases from controls, with an AUC=1 indicating a perfect test. The 95% CI and p-values for the AUC curves were also calculated.

Partial least squares—discriminant analysis (PLS_DA) is a further method that was used to enhance the separation between the groups. The PCA components are rotated such that maximum separation between the groups is obtained (Wishart D S., Methods Mol Biol. 2010; 593:283-313). Permutation analysis was used to confirm that the separation achieved between groups was not due to chance but was statistically significant. Random relabeling of the metabolomic data is performed thousands of times and PLS_DA was systematically repeated.

Genetic computing was also used as another statistical tool. Genetic computing is thought to be superior to conventional statistical analysis in explaining the differences between healthy and diseased individuals and in finding the most significant and interesting differences between groups. It generates rules by which an optimal number of variables can be selected from a large number of exploratory variables e.g. metabolite concentrations, and also optimally selects the interactions between these variables for the prediction of the outcomes of interest such as the presence or absence of preeclampsia. A highly accurate classification of individuals into disease and non-disease groups is generated. TheGmax computer program version 11.09.23 (www.thegmax.com) was used for evolutionary computing analysis.

The above-mentioned techniques were used in the examples below.

### Example 1

Blood plasma was collected prospectively in women between 11⁺⁰ and 13⁺⁶ weeks gestation at King's College Hospital, London, UK. Serum was stored at −80° C. and subsequently transferred in a frozen state to the testing lab. The definition of preeclampsia used was based on the International Society for the Study of Hypertension in Pregnancy (Brown M A et al. The classification and diagnosis of hypertensive disorders of pregnancy: Statement from the International Society of Hypertension in Pregnancy (ISSHP). Hypertension Pregnancy 2001; 20: ix-xv). Based on this definition, systolic blood pressure should be 140 mmHg or more and/or the diastolic blood pressure should be 90 mmHg or more on at least two occasions 4 hours apart and developing after 20 weeks gestation in a previously normotensive woman. In addition, there should be a significant amount of protein in the urine (proteinuria) defined as ≧300 mg in the total volume of urine collected over a 24 hour period. Alternatively, significant proteinuria was defined as at least 2+ based on a semi-quantitative measurement using urine dipstick from a specimen of mid-stream urine or a catheter urine specimen if a 24 hour urine collection is not available.

For purposes of the study, early-onset preeclampsia was defined as preeclampsia developing and requiring delivery before or at 34 weeks gestation while late-onset preeclampsia was defined as cases developing preeclampsia after 34 weeks gestation. Neither study subjects nor control subjects had major birth defects, such as Down syndrome. A single specimen per patient was used.

Study and control cases were limited to those with a CRL of 45-84 mm (11⁺⁰ to 13⁺⁶) gestation at the time of sample collection.

Maternal weight and metabolite concentrations as listed in Tables 1 and 2 were entered in a logistic regression model for estimating the probabilities of early and late-onset preeclampsia.

TABLE 1
Concentrations of Metabolites: Early-
Onset Preeclampsia vs. Controls

| | Early-Onset | | |
|---|---|---|---|
| | Preeclampsia | Controls | |
| Metabolite | Mean (SD) | Mean (SD) | p value |
| Acetate | 20.0 (7.0) | 53.6 (56.9) | <0.001 |
| Alanine | 264.3 (52.7) | 355.1 (213.3) | 0.01 |
| Arginine | 108.4 (16.6) | 126.8 (31.2) | 0.03 |
| Choline | 10.9 (3.1) | 183.7 (322.8) | 0.001 |
| Ethanol | 39.0 (29.7) | 58.1 (30.6) | 0.04 |
| Formate | 12.4 (6.0) | 20.8 (16.8) | 0.007 |
| Glycerol | 159.8 (54.1) | 399.6 (450.8) | 0.001 |
| Glycine | 190.3 (47.1) | 260.5 (118.4) | 0.002 |
| Leucine | 64.2 (15.6) | 108.4 (86.3) | 0.002 |
| Methanol | 204.4 (81.6) | 267.4 (136.2) | <0.04 |
| Methionine | 20.2 (4.8) | 24.7 (7.8) | <0.04 |
| Ornithine | 30.4 (12.7) | 40.4 (17.3) | 0.02 |
| Phenylalanine | 61.3 (12.7) | 86.6 (48.6) | 0.003 |
| Propylene glycol | 11.2 (6.2) | 7.7 (3.5) | 0.01 |
| Serine | 111.7 (26.7) | 167.9 (94.8) | 0.001 |
| Succinate | 4.4 (1.9) | 12.9 (15.4) | 0.001 |
| Threonine | 123.4 (24.0) | 157.9 (66.9) | 0.005 |

TABLE 2
Concentrations of Metabolites: Late-
Onset Preeclampsia vs. Controls

| | Late-Onset | | |
|---|---|---|---|
| | Preeclampsia | Controls | |
| Metabolite | Mean (SD) | Mean (SD) | p value |
| 2-Hydroxybutyrate | 34.3 (15.9) | 22.0 (10.1) | 0.009 |
| Acetamide | 6.7 (3.2) | 10.8 (7.0) | 0.003 |
| Acetate | 105.2 (124.8) | 53.7 (56.9) | 0.03 |
| Acetone | 29.6 (11.2) | 16.2 (7.2) | <0.001 |
| Carnitine | 46.9 (17.9) | 32.5 (15.7) | 0.004 |
| Creatine | 47.0 (17.4) | 38.0 (14.3) | <0.05 |
| Creatinine | 66.6 (12.7) | 56.4 (15.2) | 0.02 |
| Dimethylamine | 2.5 (1.2) | 3.4 (1.8) | <0.05 |
| Glucose | 4843.8 (1799.6) | 3716.9 (701.6) | <0.03 |
| Glycerol | 1090.7 (444.9) | 399.6 (450.8) | <0.001 |
| Propylene glycol | 10.7 (5.7) | 7.7 (3.5) | <0.02 |
| Trimethylamine | 7.2 (5.5) | 12.1 (2.9) | <0.001 |
| Methylhistidine | 82.1 (38.3) | 49.8 (14.5) | 0.004 |

Using the traditional logistic regression approach, a parsimonious model using only formate and propylene-glycol blood concentrations was developed. This had a 79.3% accuracy for distinguishing normal control patients from early-onset preeclampsia cases.

Table 3 shows the numbers of cases in the early and late-onset preeclampsia groups, and also in the control group. In addition, the mean (SD) fetal crown-rump length (CRL) in milimeters at maternal blood collection is shown.

TABLE 3
Preeclampsia and Control Cases: CRL at Specimen Collection

| Patient Category | Number of Cases | CRL Mean (SD) |
|---|---|---|
| Early-Onset Preeclampsia | 16 | 61.3 (7.3) |
| Late-Onset Preeclampsia | 16 | 62.1 (9.0) |
| Normal Controls | 43 | 64.8 (8.8 |

In a similar fashion, using the traditional logistic regression approach, a model for predicting late-onset preeclampsia cases was developed. In some of the calculations, the significant predictors in the model were acetone, trimethymine, acetamide, glycerol, glutamine, acetone and creatinine. The model had a 100% accuracy for separating late-onset preeclampsia from normal cases. Data analyses were performed using the GMAX computer program. Table 4 shows the area under the ROC curve for the prediction of early-onset and late-onset preeclampsia using the logistic regression statistical approach.

TABLE 4
Receiver Operating Characteristics Curve
(ROC) for Prediction of Preeclampsia

| | Area Under ROC | |
|---|---|---|
| Preeclampsia Category | (95% CI) | p value |
| Early-onset | 0.79 (0.70, 0.91) | 0.001 |
| Late-onset | 0.82 (0.71, 0.93) | <0.001 |

Based on traditional logistic regression prediction of probability

Analyses of diagnostic accuracy were also performed using Genetic computing (TheGmax, version 10.10.22, U.K. http://www.thegmax.com/). Modeling based on metabolite concentrations alone had high accuracy for prediction of early- and late-onset preeclampsia. The combination of betaine, propylene-glycol, acetate, acetone and valine concentrations gave an area under the ROC curve (AUC)=1.00 for the prediction of early-onset preeclampsia. Furthermore, a parsimonious model based only on the concentration of propylene-glycol gave an AUC=0.88 as shown in Table 5.

TABLE 5
Prediction of Early-Onset Preeclampsia Based on
Metabolites and CRL: Genetic Computing Analysis

| Marker Combination | Area Under ROC Curve |
|---|---|
| Creatine, Choline, CRL | 1.0 |
| CRL, Creatine | 0.86 |

For late-onset preeclampsia, algorithms consisting of multiple metabolites or a parsimonious model of only a single metabolite such as glycerol had a high diagnostic accuracy as shown by the areas under the ROC curves in Table 6.

TABLE 6
Prediction of Late-Onset Preeclampsia Based ONLY on
Metabolite Concentration: Genetic Computing analysis

| Marker Combination | Area Under ROC Curve |
|---|---|
| Multiple Metabolites (Glycerol, ethylene | 1.0 |
| glycol, threonine, carnitine, and alanine) | |
| Glycerol Only | 0.95 |

As can be seen from the foregoing, both traditional statistical analysis and newer genetic computing approaches show that using only metabolites identified and quantified in the first trimester, such as before 14 weeks, accurate early prediction of both early-onset and late-onset preeclampsia can be accomplished.

### Example 2

Gestational age of the pregnancy represented by CRL was evaluated with metabolite concentrations for predicting the early-onset and late-onset preeclampsia risk and calculating the diagnostic accuracy of the predictive algorithms. The statistical methods used are the same as the ones described in Example 1, namely, logistic regression analyses, ROC curves, calculation of area under the curves, and genetic computing. Using logistic regression modeling techniques, one of the predictors of early-onset preeclampsia was propylene glycol with formate making a borderline contribution to predictive accuracy. With this particular metabolite combination, gestational age information as represented by CRL measurements did not contribute significantly to prediction beyond these two biomarkers. Thus, precise knowledge of gestational age may not be needed to predict early-onset preeclampsia.

The combination of trimethylamine, acetamide, glycerol, glutamine and creatinine concentrations had a 100% accuracy for separating those destined to develop late-onset preeclampsia compared to cases that ultimately did not develop any preeclampsia (normal group).

Based on genetic computing, a combination of creatine and choline concentrations, and CRL was highly accurate (AUC 1.00) for predicting early-onset preeclampsia. A parsimonious model, which combined CRL and creatine concentration also accurately predicted early-onset preeclampsia as shown in Table 5. With these metabolite combinations, CRL did not contribute significantly to the prediction of the late-onset preeclampsia.

### Example 3

As known in the art, Doppler ultrasound measurement of blood flow resistance in the uterine arteries of pregnant women obtained in the first trimester (11⁺⁰ to 13⁺⁶ weeks) can help to predict the subsequent development of preeclampsia later in the same pregnancy (Poon L. C. et al., Prenat Diag 2010; 30:216-23; Akolekar R., et al., Prenatal Diagn 2009; 29:753-60, 847-51).

In this example, estimation (calculations) and prediction of preeclampsia risk were performed using metabolite concentrations in maternal blood, gestational age information and the first trimester uterine artery Doppler ultrasound information. Doppler blood flow velocity measurement was obtained and recorded as previously described in the published literature (Poon L C Y et al. Hypertensive disorders in pregnancy: Screening by uterine artery Doppler at 11-13 weeks. Ultrasound Obstet General 2009; 34:142-8). Measurements were made in both the left and right maternal uterine artery. A widely employed index of measurement, the Pulsatility Index (PI) was used to quantitate the resistance to blood flow in the vessel. The average of both the left and right was used in these calculations. A high PI indicates increased resistance to blood flow in the vessels that feed the uterine artery and the placental vessels, usually due to constriction or blockage of these same placental vessels. Spasm or blockage in placental vessels is known to be a feature of preeclampsia. Each measured value in the unaffected normal group and preeclampsia group was converted to multiple of the median (MOM) values after adjustment for racial origin, gestational age, maternal age, and body mass index (BMI), which is an index of maternal weight after standardizing for height. Measurements were made from both the left and right uterine artery and for each patient the average of the two uterine artery Doppler PI values expressed in MOM (L-PI) was used for preeclampsia prediction. The statistical methods as described in EXAMPLES 1 and 2 were used. Maternal weight, metabolite concentrations and uterine artery Doppler measurements were considered in this prediction model. In some of the calculations, the significant predictors of early-onset preeclampsia development were threonine and uterine artery Doppler PI (UtPI). The probability of an individual developing early-onset preeclampsia based on the algorithm is given by the following logistic equation: Early-onset Preeclampsia=1/[1+e^{-(-0.40×threonine+4.018×UtPI)}] where −0.40 and 4.018 represent the β-coefficients for threonine and uterine artery Doppler PI respectively. The measured values for threonine and UtPI (MOM) obtained were plugged into the equation as shown. UtPI contributed to the prediction of early-onset preeclampsia using this statistical approach. The accuracy for distinguishing early-onset preeclampsia from normal controls was 81.6%. UtPI was not significantly different between late-onset preeclampsia and normal control groups: means (SD), UtPI (MOM) values[1.18 (0.34) vs. 1.05 (0.29)], p=0.21 respectively. When using the metabolites as described above, UtPI was not a significant predictor of late-onset preeclampsia when added to the logistic regression analysis as shown in Table 7.

TABLE 7
Receiver Operating Characteristics Curve Prediction
of Preeclampsia*: Gestational Age and Uterine Doppler
Artery Measurements [Ut PI (MOM)] Considered

| | Area Under ROC Curve | |
|---|---|---|
| Preeclampsia Category | (95% CI) | p Value |
| Early-onset** | 0.90 (0.8, 1.0) | <0.001 |
| Late-onset*** | — | — |

*Traditional logistic regression approach
**Threonine and UtPI(MoM) were the significant predictors
***UtPI (MOM) did not contribute significantly to the prediction of late-onset preeclampsia

The analysis of the results using Genetic computing yielded the following results. A complex model consisting of the concentrations of acetate, dimethylamine, acetamide, UtPI (MOM), succinate, trimethylamine, glutamine, citrate and ornithine had total area under the ROC curve of 1.0 for the prediction of early-onset preeclampsia, as shown in Table 8. The percentage contribution of each of these markers to the predictive accuracy were as follows: acetate 54.2%, UtPI (MOM) 28.3%, ornithine 7.7%, with the other markers contributing less than 5% each to the discrimination.

Including UtPI (MOM) information did not contribute to the accuracy of prediction of late-onset preeclampsia using genetic computing methods of analysis as shown in Table 8.

TABLE 8
Receiver Operating Characteristics (ROC) Curve for
Prediction of Preeclampsia^{†}: Uterine Artery
Doppler [UtPI (MOM)] considered: Genetic computing

| Preeclampsia Category | Area Under ROC Curve |
|---|---|
| Early*-Onset Preeclampsia | 1.0 |
| Late**-Onset Preeclampsia | — |

^{†}logistic regression approach
*Significant predictors: acetate, dimethylamine, acetamide, UtPI (MOM), succinate, trimethylamine, glutamine, citrate, and ornithine
**UtPI (MOM) did not contribute to prediction of late-onset preeclampsia

A combination of glycerol, choline and alanine concentrations alone generated an ROC curve with area of 1.0 for prediction of late-onset preeclampsia. The percentage contributions of each of these metabolites to preeclampsia prediction were 84.7%, 11.1% and 4.2% respectively.

### Example 4

The data for this study were derived from prospective screening for adverse obstetric outcomes in women attending their routine first-hospital-visit in pregnancy. In this visit, which was held at 11⁺⁰-13⁺⁶ weeks gestation, maternal characteristics and medical history were recorded, an ultrasound scan was performed to determine gestational age from the fetal crown-rump length and to diagnose major fetal abnormalities. Doppler ultrasound was also carried out to measure the uterine artery pulsatility index (PI) bilaterally and record the average of the two PI values. Serum samples were also obtained and stored at −80° C. for subsequent laboratory analysis. The women were screened over a 42 month period and they all gave written consent to participate in the study, which was approved by the King's College Hospital research ethics committee.

Metabolomic studies were carried out in 30 singleton pregnancies that subsequently developed early-PE requiring delivery before 34 weeks and 60 unaffected controls. The cases were drawn from the screening study population. The controls were also from the same population from pregnancies with no complications and normal outcome matched to the cases for storage time. Pertinent maternal and fetal characteristics in the early-PE and controls are compared in Table 9.

TABLE 9
Comparison of the early preeclampsia and control groups

| | Early- | | | | p- |
|---|---|---|---|---|---|
| Parameter | preeclampsia | | Control | | value |
| Maternal age in | 30.6 | (6.8) | 31.8 | (5.8) | 0.41 |
| years, mean (SD) | | | | | |
| Racial origin, n (%) | Number (%) | | Number (%) | | 0.13 |
| White | 32 | (52.5) | 10 | (34.5) | |
| Black | 22 | (36.1) | 16 | (55.2) | |
| Asian | 7 | (11.5) | 2 | (6.9) | |
| Other | 0 | (0) | 1 | (3.4) | |
| Nullipara (%) | 47.5 | | 41.4 | | 0.65 |
| Weight in kg, | 72.4 | (17.0) | 66.8 | (14.9) | 0.10 |
| mean (SD) | | | | | |
| Crown-rump length | 63.3 | (8.6) | 65.4 | (8.5) | 0.28 |
| in mm, mean (SD) | | | | | |
| Uterine pulsatility | 1.5 | (0.5) | 0.95 | (0.3) | <0.001 |
| index (MoM), | | | | | |
| mean (SD) | | | | | |

MoM = multiples of median for gestational age

The only significant difference was in uterine artery PI which was higher in the PE group. A total of 42 metabolites were identified and quantified in the maternal plasma samples, and significant differences between the early-PE cases and controls were found for 20 of the metabolites (Table 10).

TABLE 10
Metabolite concentrations in early preeclampsia and controls

| | Early PE | Controls | | |
|---|---|---|---|---|
| Metabolite | Mean (SD) | Mean (SD) | p-value | |
| Hydroxy- | 21.4 (5.6) | 19.5 (7.4) | 0.25 | |
| butyrate_2 | | | | |
| Hydroxy- | 31.6 (19.1) | 27.0 (16.2) | 0.44 | |
| butyrate_3 | | | | |
| Hydroxyiso- | 9.2 (1.7) | 7.6 (3.1) | 0.01 | |
| valerate_3 | | | | |
| Acetamide | 9.6 (5.4) | 10.0 (6.3) | 0.96 | |
| Acetate | 17.9 (6.0) | 48.4 (50.4) | <0.001 | (<0.001) |
| Acetoacetate | 14.5 (5.1) | 18.8 (9.7) | 0.59 | |
| Acetone | 14.5 (3.6) | 17.2 (20.3) | 0.57 | |
| Alanine | 263.8 (51.5) | 332.1 (184.2) | 0.02 | |
| Arginine | 113.4 (16.5) | 124.2 (28.8) | 0.056 | |
| Asparagine | 36.5 (17.2) | 34.5 (14.5) | 0.48 | |
| Betaine | 29.2 (6.9) | 25.8 (8.8) | 0.31 | |
| Carnitine | 30.2 (6.3) | 31.8 (14.1) | 0.70 | |
| Choline | 10.4 (2.6) | 143.9 (277.4) | 0.001 | (<0.001) |
| Citrate | 85.9 (19.1) | 79.3 (19.3) | 0.06 | |
| Creatine | 34.7 (10.6) | 37.4 (13.0) | 0.06 | |
| Creatinine | 56.8 (11.8) | 56.7 (14.5) | 0.74 | |
| Dimethyl- | 3.5 (1.4) | 4.0 (2.7) | 0.219 | |
| amine | | | | |
| Ethanol | 33.1 (22.3) | 49.3 (30.2) | 0.08 | |
| Formate | 12.4 (4.5) | 19.3 (14.8) | 0.032 | |
| Glucose | 4194.1 (1229.6) | 3702.2 (713.5) | 0.342 | (0.019) |
| Glutamine | 331.0 (57.2) | 288.2 (82.0) | 0.007 | |
| Glycerol | 166.5 (42.5) | 484.2 (341.4) | <0.001 | |
| Glycine | 188.0 (42.8) | 239.6 (106.1) | 0.001 | (0.035) |
| Isobutyrate | 6.3 (2.1) | 6.3 (2.1) | 1.0 | |
| Isoleucine | 37.6 (9.6) | 46.2 (19.3) | 0.023 | (0.022) |
| Isopropanol | 5.9 (4.1) | 38.4 (98.8) | 0.015 | (0.034) |
| Lactate | 1006.2 (396.8) | 1035.6 (505.9) | 0.78 | |
| Leucine | 67.5 (15.5) | 96.9 (74.9) | 0.003 | |
| Malonate | 16.8 (6.7) | 18.9 (9.8) | 0.34 | |
| Methionine | 20.6 (4.2) | 23.5 (7.1) | 0.027 | (0.024) |
| Ornithine | 36.1 (11.9) | 38.2 (15.7) | 0.18 | |
| Phenyl- | 62.9 (12.1) | 79.6 (42.7) | 0.002 | |
| alanine | | | | |
| Proline | 138.6 (52.0) | 142.6 (58.5) | 0.067 | |
| Propyl- | 9.9 (4.9) | 8.0 (3.4) | 0.035 | (0.04). |
| ene_glycol | | | | |
| Pyruvate | 89.3 (27.0) | 71.3 (26.7) | 0.026 | (0.006) |
| Serine | 115.5 (23.8) | 150.3 (84.3) | 0.004 | |
| Succinate | 4.3 (1.6) | 12.4 (13.9) | 0.007 | |
| Threonine | 127.1 (29.0) | 145.9 (60.3) | 0.025 | |
| Trimethyl- | 13.3 (2.0) | 11.2 (3.1) | 0.001 | (0.008) |
| amine | | | | |

| | | | |
|---|---|---|---|
| Tyrosine | 54.1 (15.3) | 58.0 (22.3) | 0.21 |
| Valine | 119.1 (29.5) | 129.8 (51.1) | 0.22 |
| Methyl- | 50.7 (12.9) | 50.0 (14.6) | 0.75 |
| histidine | | | |

The p-values in brackets are based on Mann-Whitney U-test for biomarkers with non-normal distributions

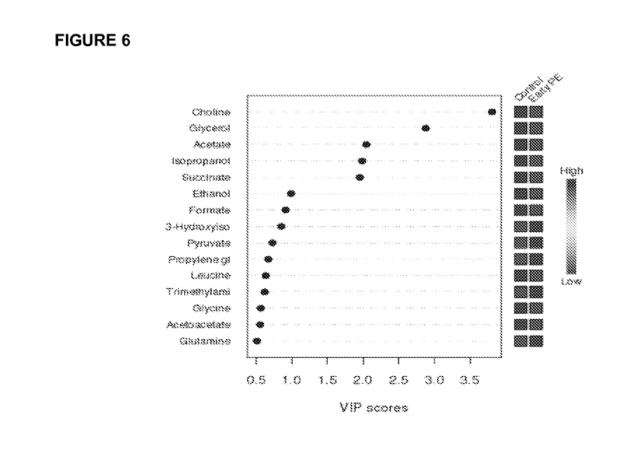

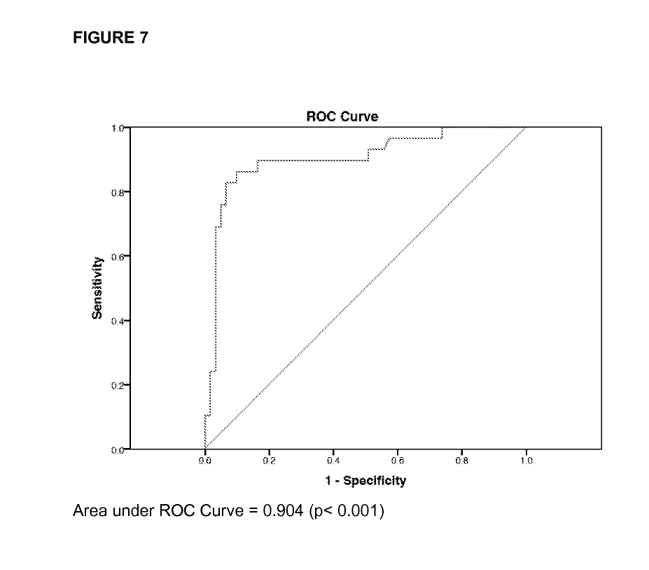

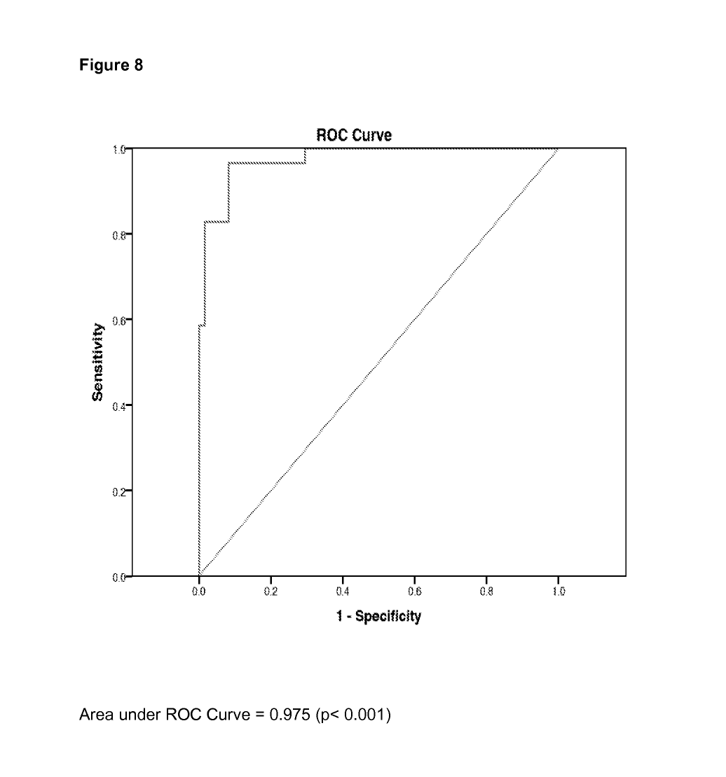

The separation between the cases of early-PE and controls from the PCA analysis of the NMR data is shown in FIG. 7. The PLS-DA analysis resulted in a clear separation between the groups (FIG. 8). Permutation testing demonstrated that the observed separation was not by chance (p<0.005). A Variable Importance in Projection (VIP) plot, in which the metabolites were ranked by their contribution to distinguishing the cases of early-PE from controls is shown in FIG. 6. The greater the distance from the Y-axis, the greater is the contribution of a particular metabolite in distinguishing cases from controls. This plot also indicates whether the metabolite concentration is increased or decreased in cases relative to controls.

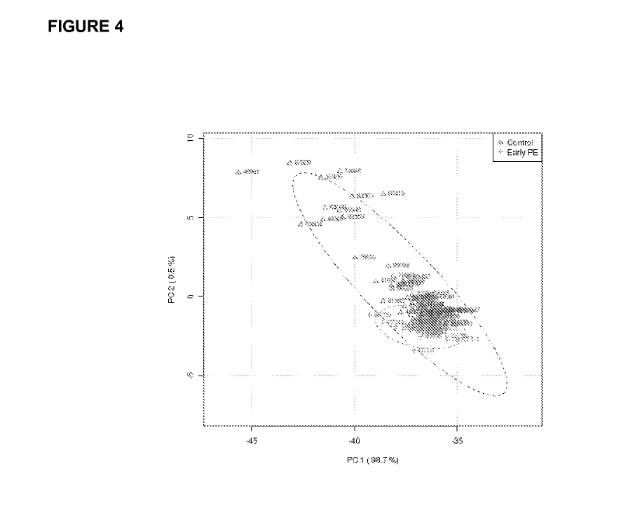

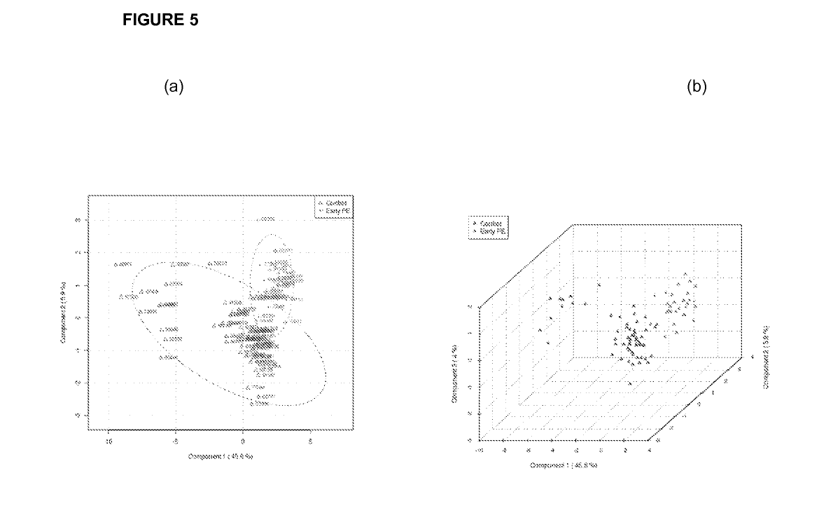

Two models were developed, using logistic regression analysis, for the prediction of early-PE; one evaluated the following independent variables: four metabolites (citrate, glycerol, hydroxyisovalerate, methionine) in combination with maternal characteristics (weight and the presence/absence of medical disorders) and another evaluated three metabolites (citrate, glycerol, hydroxyisovalerate) in combination with maternal characteristics (parity and the presence or absence of medical disorders) and uterine artery PI and fetal CRL. The estimated performance of screening for early-PE by these models is summarized in Table 11 which is based on standard statistical analysis with the corresponding ROC curves without and with inclusion of Doppler measurements demonstrated in FIGS. 4 and 5 respectively. As seen in Table 11, the first model (without Ut-PI) resulted in the estimated detection rate of early-PE of 75.9%, at a false positive rate of 4.9% and the respective values for the second model which included uterine Doppler PI were 82.6% and 1.6%. Ut PI by itself had a 40% detection rate at 8.2% false positive rate.

TABLE 11
Estimated performance of screening for early preeclampsia

| Regression | AUROC | Sensitivity | FPR |
|---|---|---|---|
| model | (95% CI) | (%) | (%) |
| Metabolites* | 0.904 (0.828, 0.98) | 75.9 | 4.9 |
| Metabolites**, uterine | 0.98 (0.95, 1.00) | 82.6 | 1.6 |
| artery PI and fetal CRL | | | |

*Citrate, glycerol, hydroxyisovalerate, methionine
**Acetate, glycerol, hydroxyisovalerate
AUROC = area under the receiver operating characteristic curve
CI = confidence interval
FPR = false positive rate
PI = pulsatility index
CRL = crown-rump length

The results of genetic computing analysis using a minimum (parsimonious model) number of predictors are shown in Table 12. Two models were developed, one using metabolites (glutamine, pyruvate, propylene glycol, trimethylamine, hydroxy butyrate) in combination with maternal characteristics (weight and medical disorder) and another using metabolites (glutamine, pyruvate, propylene glycol, trimethylamine, hydroxybutyrate, carnitine, hydroxyisovalerate) in combination with uterine artery PI. The diagnostic sensitivities at low false positive rates are shown in Table 12.

TABLE 12
Estimated performance of screening for early
preeclampsia using genetic computing analysis

| Regression | | Sensitivity | FPR |
|---|---|---|---|
| model | AUROC | (%) | (%) |
| Metabolites*, maternal weight | 0.84 | 50 | 5.0 |
| and medical disorders | | | |
| Metabolites**, uterine artery PI | 0.94 | 60 | 3.0 |

*glutamine, pyruvate, propylene glycol, trimethylamine, hydroxy butyrate (parsimonious model)
**pyruvate, propylene glycol, trimethylamine, hydroxy isovalerate-3 (parsimonious model)
AUROC = area under the receiver operating characterist curve
FPR = false positive rate

The model that excluded uterine Doppler measurements also had a 70% detection rate at 15% false positive rate, while including uterine Doppler measurements increased detection rate to 90% while simultaneously lowering the false positive rate to 11%. Complex models using a larger number of predictors achieved even greater areas under the ROC curves and thus improved diagnostic accuracy (not shown).

### Example 5

The women were screened over a 42 month period and they all gave written consent to participate in the study, which was approved by the King's College Hospital research ethics committee. Briefly, women were recruited at 11^{+o}-13⁺⁶ weeks gestation. Maternal characteristics and medical history were documented and first trimester ultrasound including CRL and uterine artery Doppler pulsatility index (PI) were measured. The average of the left and right PI values was used for this analysis. Maternal serum samples were also obtained and stored at −80° C. for subsequent laboratory analysis.

A total of 30 singleton pregnancies that subsequently developed late-onset PE requiring delivery after 34 weeks, formed the study group and were matched with 60 unaffected controls. Results for a total of 30 cases and 59 controls are reported as insufficient volume of serum was available for metabolomic analysis in one of the control samples. Table 13 compares the maternal age, weight, race and gestational age at blood collection based the crown-rump length (CRL) measurements, between late-onset preeclampsia and normal case.

TABLE 13
Demographic and other characteristics:
Late preeclampsia versus control group

| | | | |
|---|---|---|---|
| | Late- | | p- |
| Parameter | preeclampsia | Control | value |

| | | | |
|---|---|---|---|
| Number of cases | 30 | 59 | — |

| | | | | | |
|---|---|---|---|---|---|
| Maternal age | 31.2 | (6.4) | 30.8 | (5.6) | 0.81 |
| in years, mean (SD) | | | | | |

| | | | |
|---|---|---|---|
| Racial origin, n (%) | Number (%) | Number (%) | 0.02 |

| | | | | | |
|---|---|---|---|---|---|
| White | 14 | (46.7) | 44 | (74.6) | |
| Black | 14 | (46.7) | 14 | (23.7) | |
| Asian | 0 | (0) | 1 | (1.7) | |
| Mixed | 2 | (6.7) | 0 | (0) | |
| Nullipara (%) | 12 | (40) | 31 | (52.5) | 0.37 |
| Weight in kg, | 74.9 | (15.7) | 67.7 | (12.2) | 0.03 |
| mean (SD) | | | | | |
| Crown-rump length | 62.0 | (9.1) | 62.7 | (7.6) | 0.69 |
| in mm, mean (SD) | | | | | |
| Uterine pulsatility | 1.07 | (0.35) | 0.98 | (0.31) | 0.22 |
| index (MoM), | | | | | |
| mean (SD) | | | | | |

There was a significant difference in race with a lower percentage of whites and higher percentage of blacks in the late preeclampsia group as well as greater body weight in the preeclampsia group as seen in Table 13. The mean (SD) metabolite concentrations were compared between the two groups. A total of 17 metabolites were present in significantly different concentrations in late-preeclampsia versus the control groups (Table 14). Fourteen of these 17 metabolite concentrations were increased in late-onset preeclampsia groups while 3 were reduced.

TABLE 14

| | Early PE | Controls | |
|---|---|---|---|
| | Mean (SD) | Mean (SD) | |
| | (units of | (units of | |
| Metabolite | concentration) | concentration) | p-value |
| Hydroxybutyrate_2 | 28.0 (14.4) | 21.2 (7.5) | 0.02 |
| Hydroxybutyrate_3 | 49.9 (46.7) | 29.7 (19.1) | 0.038 |
| Hydroxyisovalerate_3 | 6.5 (3.3) | 4.7 (2.5) | 0.008 |
| Acetamide | 11.9 (7.8) | 16.1 (6.4) | 0.008 |
| Acetate | 80.6 (101.5) | 49.1 (52.5) | 0.12 |
| Acetoacetate | 18.9 (9.8) | 16.5 (9.6) | 0.27 |
| Acetone | 22.1 (11.4) | 14.9 (8.5) | 0.003 |
| Alanine | 366.8 ((2.4.8) | 323.8 (151.2) | 0.27 |
| Arginine | 136.3 (55.5) | 131.2 (35.9) | 0.65 |
| Asparagine | 31.3 (11.3) | 32.4 (13.7) | 0.71 |
| Betaine | 33.3 (23.6) | 21.6 (9.4) | 0.14 |
| Carnitine | 46.8 (24.8) | 27.8 (20.0) | 0.001 |
| Choline | 172.3 (341.5) | 185.7 (351.6) | 0.87 |
| Citrate | 85.9 (26.9) | 74.1 (23.5) | 0.028 |
| Creatine | 41.5 (5.9) | 33.4 (15.6) | 0.024 |
| Creatinine | 63.2 (16.5) | 55.1 (14.7) | 0.021 |
| Dimethylamine | 3.2 (1.7) | 4.4 (2.1) | 0.012 |
| Ethanol | 67.7 (42.6) | 56.1 (37.2) | 0.19 |
| Formate | 27.0 (13.8) | 29.0 (17.8) | 0.60 |
| Glucose | 4312.9 (1783.0) | 3362.4 (765.9) | 0.008 |
| Glutamine | 253.1 (131.1) | 218.5 (66.9) | 0.182 |
| Glycerol | 800.7 (541.7) | 312 (296.8) | <0.001 |
| Glycine | 238.4 (129.3) | 244.0 (115.7) | 0.84 |
| Isobutyrate | 7.6 (2.5) | 7.6 (3.2) | 1.0 |
| Isopropanol | 10.7 (4.6) | 7.7 (4.8) | 0.006 |
| Lactate | 1213.1 (564.7) | 1100.9 (689.3) | 0.44 |
| Leucine | 114.5 (98.5) | 87.1 (61.9) | 0.112 |
| Malonate | 23.1 (14.3) | 23.1 (8.7) | 0.97 |
| Methionine | 24.7 (7.4) | 23.6 (6.5) | 0.48 |
| Ornithine | 36.8 (17.4) | 42.3 (22.5) | 0.24 |
| Phenylalanine | 78.0 (45.9) | 80.9 (45.4) | 0.78 |
| Proline | 172.2 (57.7) | 165.7 (56.4) | 0.61 |
| Propylene_glycol | 11.1 (5.0) | 11.8 (4.9) | 0.51 |
| Pyruvate | 83.1 (45.8) | 62.1 (24.1) | 0.006 |
| Serine | 148.4 (103.4) | 158.6 (92.2) | 0.635 |
| Succinate | 13.2 (13.8) | 13.4 (12.9) | 0.9 |
| Threonine | 157.2 (60.2) | 166.2 (62.5) | 0.5 |
| Trimethylamine | 6.03 (2.0) | 7.6 (3.3) | 0.005 |
| Tyrosine | 65.1 (23.7) | 62.3 (21.7) | 0.6 |
| Valine | 142.5 (50.6) | 121.6 (43.3) | <0.05 |
| Methylhistidine | 70.3 (40.0) | 38.9 (20.3) | <0.001 |

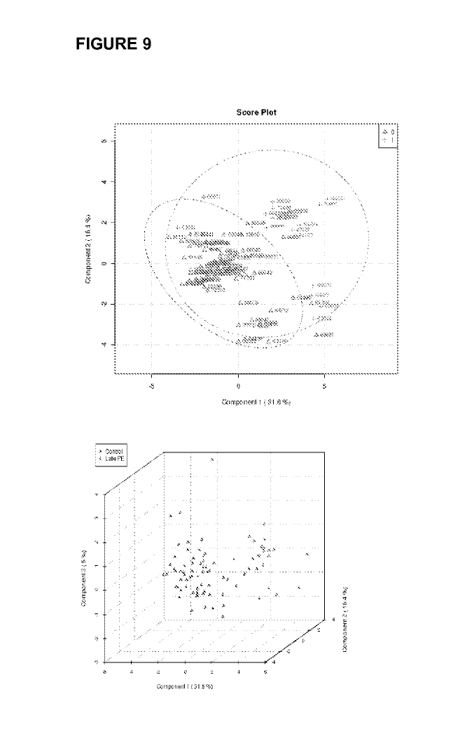

In FIG. 9a, the PCA plot shows separation between late-onset preeclampsia (in green) group compared to normal cases (in red). The two principal components (metabolite set) accounted for 31.6% (on x-axis) and 16.4% (on y-axis) of the separation of the late-preeclampsia from the control group.

The PLS-DA plot is shown in FIG. 9b. Permutation testing based on 2000 repeat samplings revealed that the observed separation of late onset preeclampsia from the normal group achieved by NMR was statistically highly significant (p<0.0005) and not due to chance.

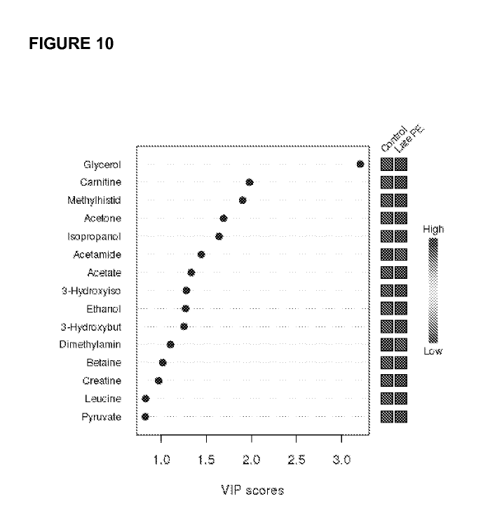

FIG. 10 displays the Variable Importance in Projection (VIP) plot. Here the metabolites were ranked in descending order based on their discriminating power. The greater the distance on the X-axis, the greater the contribution of a particular metabolite in distinguishing late preeclampsia from control group. A heat map is shown on the right of the VIP plot. Red indicates that the particular metabolite concentration is increased in late preeclampsia cases while green indicates reduced concentration in preeclampsia.

Forced entry logistic regression analysis was performed considering only maternal weight, medical problems and metabolite concentrations as independent predictors for late-onset preeclampsia. A parsimonious model consisting of a combination of maternal weight (p=0.05), glycerol (p<0.05), trimethylamine (p=0.029), valine (p=0.026) and methylhistidine (0.025) had a sensitivity of 93.1% and specificity 98.3% for the prediction of late onset preeclampsia and an overall a accuracy of 96.6% for the correct identification of preeclampsia status (Table 15).

TABLE 15
Biomarker models for prediction of late onset
of preeclampsia: Logistic regression analyses

| Predictive | Sensitivity | Specificity |
|---|---|---|
| model | (%) | (%) |
| Model 1* | 93.1 | 98.3 |
| Model 2** | 86.7 | 96.6 |
| Model 3*** | 70.0 | 96.6 |

*hydroxybutyrate-3, glycerol, trimethylamine, valine and methylhistidine
**maternal race, weight, glycerol, pyruvate, trimethylamine, valine, methylhistidine
***maternal race, glycerol, trimethylamine, valine and methylhistidine

A similar analysis was performed using stepwise forward entry regression. The significant predictors were race (p<0.005), glycerol (p=0.009), pyruvate (p=0.04), trimethylamine (p=0.017), valine (p=0.014) and methylhistidine (p=0.017). The sensitivity was 86.7% and specificity 96.6% for the prediction of late onset preeclampsia (Table 15). Overall, a 93.3% accuracy for the correct identification of preeclampsia status was achieved.

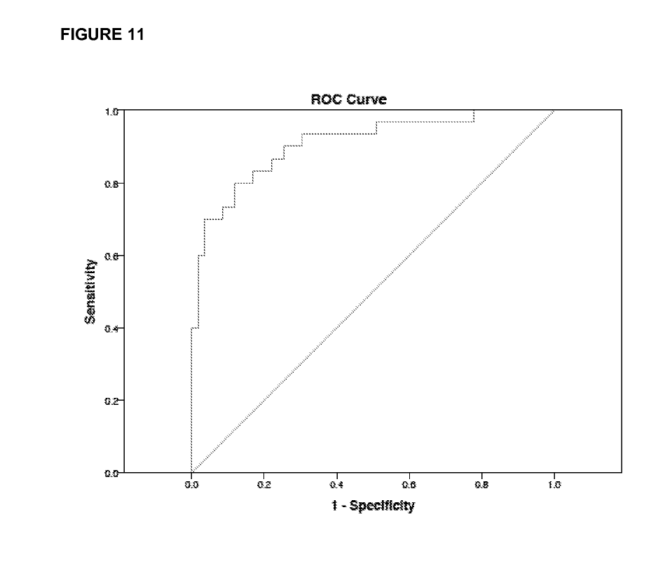

Likelihood ratios were used to estimate individual probabilities of late-onset preeclampsia based on maternal race, weight, medical problems and metabolite concentrations. These data were used to construct an ROC curve. The area under the curve (95% CI) was 0.908 (0.839, 0.977), p<0.001 is shown in FIG. 11. A detection rate of 70% was achieved at the cost of a false positive rate of 3.4% using this logistic regression approach (Table 15). The consideration of first trimester uterine artery Doppler measurements did not improve the model for late-onset preeclampsia prediction (area under the curve (95% CI), 0.908 (0.84, 0.977), p<0.001).

Using genetic analysis, uterine artery Doppler did not contribute significantly to late-preeclampsia prediction. Two prediction models were developed using this technique of statistical analysis. First, a parsimonious model was examined that used a minimum number of predictor variables and a complex model using an expanded number of predictors.

The parsimonious model had an area under the ROC curve of 0.89, with sensitivities of 50% at 99% specificity and 80% at 90% specificity. The marginal contribution to the model of the significant predictor variables were: methylhistidine—27.9%, race—20.4% and acetamide—1.4%. The complex prediction model had an area under the ROC curve of 0.96 with a 76% sensitivity at 100% specificity. The marginal contribution to this model of the significant predictor variables were: valine—36.0%, race—16.9% and weight—36.0%. Other predictors in the model contributed marginally with pyruvate, methylhistidine, hydroxybutyrate-3, glycerol, carnitine, trimethylamine and medical disorder combined contributing only 7.23% of the prediction.

When examining whether metabolomics could discriminate cases destined to develop early-versus late-onset preeclampsia, data from Examples 4 and 5 indicate that glycerol, glucose and methanol appear to be useful metabolites for distinguishing the two types of preeclampsia.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above methods without departing from the scope of the invention, it is intended that all matter contained in the above description and shown in the accompanying drawing[s] shall be interpreted as illustrative and not in a limiting sense.

1. A method for predicting a pregnant woman's risk for developing early-onset preeclampsia, the method comprising:
a) measuring concentrations of one or more metabolites selected from the group consisting of acetate, alanine, arginine, choline, creatine, dimethylamine, acetamide, trimethylamine, glutamine, citrate, ethanol, formate, glycerol, glycine, leucine, methanol, methionine, ornithine, phenylalanine, propylene glycol, serine, succinate, hydroxy-isovalerate, pyruvate, hydroxy-buturate and threonine in the pregnant woman's bodily fluid;
b) comparing the pregnant woman's one or more metabolite concentrations to concentrations of corresponding one or more metabolites obtained from pregnant women with early-onset preeclampsia and to concentrations of the corresponding one or more metabolites obtained from pregnant women exhibiting normal blood pressure and normal protein levels in urine, wherein all metabolite concentrations are measured at same or similar gestational age; and
c) predicting the pregnant woman's risk of developing early-onset preeclampsia, wherein a statistically significant change in the concentration of the one or more metabolites between the pregnant woman and the corresponding one or more metabolites from the pregnant women exhibiting normal blood pressure and normal protein levels in urine indicates a greater probability of developing early-onset preeclampsia.

2. A method for predicting a pregnant woman's risk for developing early-onset preeclampsia, the method comprising:
measuring concentrations of one or more metabolites selected from the group consisting of acetate, alanine, arginine, choline, creatine, dimethylamine, acetamide, trimethylamine, glutamine, citrate, ethanol, formate, glycerol, glycine, leucine, methanol, methionine, ornithine, phenylalanine, propylene glycol, serine, succinate, hydroxy-isovalerate, pyruvate, hydroxy-buturate and threonine in the pregnant woman's bodily fluid;
measuring crown rump length (CRL) of the pregnant woman's fetus;
comparing the pregnant woman's one or more metabolite concentrations to concentrations of corresponding one or more metabolites obtained from pregnant women with early-onset preeclampsia and to concentrations of the corresponding one or more metabolites obtained from pregnant women exhibiting normal blood pressure and normal protein levels in urine, wherein all metabolite concentrations are standardized according to CRL average values from pregnant women with early-onset preeclampsia and pregnant women exhibiting normal blood pressure and normal protein levels in urine; and
c) predicting the pregnant woman's risk of developing early-onset preeclampsia, wherein a statistically significant change in the concentration of the one or more metabolites between the pregnant woman and the corresponding one or more standardized metabolites from the pregnant women exhibiting normal blood pressure and normal protein levels in urine indicates a greater probability of developing early-onset preeclampsia.

3. The method of claim 1, wherein the one or more metabolites are selected from the group consisting of acetate, dimethylamine, acetamide, succinate, trimethylamine, glutamine, citrate, and ornithine; the group consisting of creatine and choline; the group consisting of propylene glycol and formate; the group consisting of citrate, glycerol, hydroxy-isovalerate and methionine; the group consisting of acetate, glutamine, pyruvate, propylene glycol, trimethylamine and hydroxy-buturate; the group consisting of pyruvate, propylene glycol, trimethylamine and hydroxy-isovalerate; or creatine.

4.-9. (canceled)

10. The method of claim 1, wherein early-onset preeclampsia develops between 20 weeks and 34 weeks of gestational age.

11. The method of claim 1, further comprising the step of standardizing all metabolite concentrations according to maternal weight and/or a maternal medical disorder prior to the comparing step.

12. The method of claim 11, wherein the maternal medical disorder is selected from the group consisting of chronic hypertension and diabetes mellitus.

13. A method for predicting a pregnant woman's risk for developing late-onset preeclampsia, the method comprising:
a) measuring concentrations of one or more metabolites selected from the group consisting of 2-hydroxy-butyrate, acetate, carnitine, creatine, glucose, glycerol, alanine, valine, pyruvate and methylhistidine in the pregnant woman's bodily fluid;
b) comparing the pregnant woman's one or more metabolite concentrations to the corresponding one or more metabolite concentrations obtained from pregnant women with late-onset preeclampsia and pregnant women exhibiting normal blood pressure and normal protein levels in urine, wherein all metabolite concentrations are measured at the same or similar gestational age; and
c) predicting the pregnant woman's risk of developing late-onset preeclampsia, wherein the statistically significant change in concentration of one or more metabolites between the pregnant woman and the corresponding one or more metabolites from the pregnant women exhibiting normal blood pressure and normal protein levels in urine indicates a greater probability of developing late-onset preeclampsia.

14. (canceled)

15. The method of claim 13, wherein one or more metabolites are selected from the group consisting of glycerol, choline and alanine; the group consisting of glycerol, ethylene glycol, threonine, carnitine, and alanine; the group consisting of trimethylamine, acetamide, glycerol, glutamine and creatinine; the group consisting of 3-hydroxy-butyrate, glycerol, trimethylamine, valine and methylhistidine; the group consisting of glycerol, pyruvate, trimethylamine, valine and methylhistidine; or glycerol.

16.-20. (canceled)

21. The method of claim 13, wherein late-onset preeclampsia develops at or after 34 weeks of gestational age.

22. The method of claim 13, further comprising the step of: standardizing all metabolite concentrations according to maternal race, weight and/or a maternal medical disorder prior to the comparing step.

23. The method of claim 22, wherein the maternal medical disorder is selected from the group consisting of chronic hypertension and diabetes mellitus.

24. The method of claim 2, wherein the CRL of the pregnant woman's fetus is measured at a gestational age from 8 weeks to 13 weeks.

25. The method of claim 1, further comprising the steps of:
measuring the pregnant woman's uterine artery blood flow resistance;
comparing the pregnant woman's uterine artery blood flow resistance to the uterine artery blood flow resistances of pregnant women with early-onset preeclampsia and to the uterine artery blood flow resistances of pregnant women exhibiting normal blood pressure and normal protein levels in urine; wherein a statistically significant difference in uterine artery blood flow resistance of the pregnant woman as compared to the uterine artery blood flow resistances of pregnant women exhibiting normal blood pressure and normal protein levels in urine indicates a greater probability of developing early-onset preeclampsia.

26. The method of claim 25, wherein the uterine artery blood flow resistance is measured by Doppler ultrasound.

27. The method of claim 25 wherein the uterine artery blood flow resistance is measured at a gestational age from 8 weeks to 14 weeks.

28. (canceled)

29. The method of claim 1, wherein the bodily fluid is blood or urine.

30.-32. (canceled)

33. The method of claim 1, wherein measuring the concentrations of the one or more metabolites is performed at a gestational age from 10 weeks to 18 weeks.

34. The method of claim 33, wherein measuring the concentrations of the one or more metabolites is performed at the gestational age from 11 weeks to 14 weeks.

35.-62. (canceled)
